(19) United States
(12) Patent Application Publication
(10) Pub. No.: US 2023/0041431 A1
(43) Pub. Date: Feb. 9, 2023

(54) Efficient High-Entropy Alloys Design Method Including Demonstration and Software

(71) Applicant: University of Virginia Patent Foundation, Charlottesville, VA (US)

(72) Inventors: Joseph Poon, Charlottesville, VA (US); Jie Qi, Charlottesville, VA (US)

(73) Assignee: University of Virginia Patent Foundation, Charlottesville, VA (US)

(21) Appl. No.: 17/380,549

(22) Filed: Jul. 20, 2021

(51) Int. Cl. G16C 20/30; G16C 60/00; G16C 20/70; C22C 30/00

(52) U.S. Cl. CPC G16C 20/30; G16C 60/00; G16C 20/70; C22C 30/00; G01N 25/02

(57) ABSTRACT

Embodiments relate to a system for predicting thermodynamic phase of a material. The system includes a phase diagram image scanning processing module configured to scan a binary phase diagram for each material to be used as a component of a high-entropy alloy (HEA). The system includes a feature computation processing module configured to generate a primary feature and an adaptive feature. The primary feature is representative of a probability that the HEA will exhibit a solid solution phase and/or an intermetallic phase. The adaptive feature is representative of a factor favoring formation of a desired intermetallic HEA phase. The system includes a prediction module configured to encode the primary feature and/or the adaptive feature with thermodynamic data associated with formation of HEA alloy phases to provide an output representation of the HEA alloy phases for a material under analysis.

### STATEMENT REGARDING FEDERALLY SPONSORED RESEARCH OR DEVELOPMENT

This invention was made with Government support under Grant Nos. N00014-18-1-2621 and N00014-19-1-2420 awarded by the Department of Defense. The Government has certain rights in the invention.

### FIELD

Embodiments relate to systems and methods for predicting thermodynamic phase of a material.

### BACKGROUND INFORMATION

The discovery of a new class of metallic alloys with outstanding properties, known as High-Entropy Alloys (HEAs), is poised to change the landscape of materials research and applications fundamentally, potentially creating new products that can bring significant benefits to society. High-entropy alloys (HEAs) are alloys that are formed by mixing equal or relatively large proportions of four or more elements. The term “high-entropy” is used because the entropy increase of mixing is substantially higher when there is a larger number of elements in the mix, and their proportions are more nearly equal—i.e., there is phase stability when mixing of HEAs is done. HEAs exhibit mechanical strength, ductility, corrosion-resistance, catalytic and thermal properties, thermoelectric properties, etc., that surpass those of traditional alloys.

Generally, a compositional makeup of an HEA includes of at least four elemental components, also known as Complex Composition Alloys (CCAs), or Multi-Principal-Element Alloys (MPEAs). The high entropy of mixing of HEAs tends to stabilize alloy phases beyond the normal composition boundaries of traditional alloys¹⁻³. This unique phase stability provides unprecedented compositional flexibility for exploring new materials properties unknown in traditional alloys. HEAs have been shown to have an excellent balance of mechanical strength and ductility that exceeded traditional alloys³⁻⁵. Some promising functional properties such as corrosion-resistant⁶, catalytic⁷, thermal properties⁸, and thermoelectric properties^{9,10} that exceed or comparable to those of conventional alloys have also begun to emerge.

The HEA concept founded on the vast chemical degree of freedom and nearly inexhaustible compositional space engenders a new paradigm in alloy design and discovery. However, the combinatorial compositions of HEAs in principle can reach billions and even trillions. For example, a pool of 30 elements in the periodic table can be utilized to form 142,506 different five-component HEA systems. Further inclusion of atomic percentages can lead to billions of possible compositions. Thus, the new alloy design paradigm has also come with the fundamental challenge of how to formulate the specific alloy compositions with superior structural and functional properties in the exponentially large compositional space.

Shi et al.⁵ discusses a collection of the fundamental tensile properties at ambient temperature. The dual-phase heterogeneous lamella (DPHL) structure HEAs show the best optimization of tensile strength and ductility. Other HEAs are the products based on some of the most effective strengthening mechanisms, and they tend to show better tensile strength—ductility synergy, in comparison to traditional alloys.

The formation of high-entropy phases is primarily controlled by thermodynamic and kinetic factors. To date, studies of HEAs have focused on those with the body-centered cubic (BCC), face-centered cubic (FCC), and hexagonal closed-packed (HCP) solid-solution structures. To understand the growing number of HEAs, empirical methods that utilized atomistic and thermodynamic parameters were introduced to investigate HEA compositional regions^{11,12}. The empirical approaches were later complemented by first-principles calculation^{13,14} and Calculation of Phase Diagrams (CALPHAD)^{15,16} to shed light on the thermodynamic origin of HEA formation. Despite some progress being made in understanding the formation trend of HEAs, much of the alloy design for HEAs remains challenging. More recently, there have been increasing efforts in employing data-driven methods to exploit the growing data set of HEAs. Some initial methods included the utilization of statistical models and high-throughput (HTP) experimentation¹⁷ designed to underpin the HEA phase formation trend.

Recently, there has been increasing use of machine learning (ML) in HEA research. Several groups have employed supervised ML models to predict the HEA phase regions and properties. However, limitations exist with existing ML methods, such as available datasets and the effectiveness of selected features (i.e., descriptors) for supervised training. Despite some success in categorizing the compositional regions of some solid-solution and intermetallic phases, the predictions often failed to distinguish between specific phases¹⁸⁻²⁰. In some cases, the predictions were made for some subgroups of the HEA phases²¹⁻²⁴.

Known methods for predicting and designing HEAs can be appreciated from the following:
Yeh, Jien-Wei, High-entropy multielement alloys, US 20020159914 A1
Vecchio, Kenneth; Cheney, Justin Lee, Methods of selecting material compositions and designing materials having a target property, US 20180172611 A1
Yan, Jiayi; Olson, Gregory B., Computationally-designed transformation-toughened near-alpha titanium alloy, US 20190040510 A1
Wang, Qigui; Li, Bing; Wang, Yucong; Materials property predictor for cast Aluminum alloys, US 20160034614 A1
Gong, Jiadong; Snyder, David R.; Sebastian, Jason T.; Counts, William Arthur; Misra, Abhijeet; Wright, James A.; Ductile high-temperature molybdenum-based alloys, US 20170044646 A1
Bei; Hongbin, Multi-component solid solution alloys having high mixing entropy, US 20130108502 A1
Liu; Tzeng-Feng, Composition design and processing methods of high strength, high ductility, and high corrosion resistance FeMnAlC alloys, US 20170107588 A1
Ceder, Gerbrand; Fischer, Chris; Tibbetts, Kevin; Morgan, Dane; Curtarolo, Stefano; Systems and methods for predicting materials properties, US 20060074594 A1
Zheng, Rong; Kennedy, Peter; Tanner, Roger; Apparatus and methods for predicting properties of processed material, US 20040230411 A1
Kato, Takahiko; Kuwabara, Kousuke; Fujieda, Tadashi; Aota, Kinya; Takahashi, Isamu; Yamaga, Kenji; Satake, Hiroyuki; Murakami, Hajime; Alloy structure and method for producing alloy structure, US 20170209922 A1
Park, Eun Soo; Oh, Hyun Seok; Kim, Sangjun; Yoon, Kooknoh; Ryu, Chae Woo; High entropy alloy having TWIP/TRIP property and manufacturing method for the same, US 20170233855 A1

### SUMMARY

Embodiments relate to a system for predicting thermodynamic phase of a material. The system can include a processor in operative association with memory. The processor can include plural processing modules. The plural processing modules can include a phase diagram image scanning processing module that is configured to scan a binary phase diagram for each material to be used as a component of a high-entropy alloy (HEA). The plural processing modules can include a feature computation processing module configured to generate a primary feature and an adaptive feature. The primary feature is representative of a probability that the HEA will exhibit a solid solution phase and/or an intermetallic phase. The primary feature can include: a phase field parameter (PFPₓ) that is representative of a probability of forming phase X for the whole HEA; and a phase separation percentage (PSP) that is representative of a probability that two elements of the HEA will be separated into two different phases. The adaptive feature is representative of a factor favoring formation of a desired intermetallic HEA phase. The factor can include any one or combination of: a threshold mixing enthalpy indicating that more than one type of phase formation is possible; a threshold of total atomic percentage of components in the HEA that favors dissolution of the components in the HEA in a solid solution; a threshold ratio of concentration of phase forming elements to total atomic percentage that favors precipitation of a phase; a threshold weighted electronegativity ratio that favors formation of a phase; a threshold mixing entropy that favors disordered phase formation; or a threshold ratio of a desired element content to all transitional element content that favors formation of a phase. The plural processing modules can include a prediction module configured to encode the primary feature and/or the adaptive feature with thermodynamic data associated with formation of HEA alloy phases to provide an output representation of the HEA alloy phases for a material under analysis.

Embodiments can include a high-entropy alloy that is any one or combination of: Al3Nb47Ta18Ti20V12; Al6Nb50Ta12Ti20V6W6; Al9Nb47Ta12Ti20V6W6; Al3Nb41Ti20V18W6Zr12; Nb50Ta12Ti20W6Zr12; Nb32Ta18Ti20V24Zr6; Nb32Ti20V24W12Zr12; Al3Hf6Nb35Ta12Ti20V24; Al3Nb41Ta12Ti20V18Zr6; Al3Nb47Ta18Ti20Zr12; Al9Hf6Nb41Ti20V18W6; Al6Nb32Ta18Ti20V24; Al6Nb26Ta12Ti20V30Zr6; Al3Nb41Ta12Ti20V18Zr6; Al6Nb48Ta12Ti10W6Zr18; Al9Nb29Ti20V30W6Zr6; Al3Nb42Ta21Ti20Zr14; Al6Nb39Ta21Ti20Zr14; Nb50Ta12Ti20W6Zr12; Cr5Hf6Nb48Ta7Ti20Zr14; Cr10Hf6Nb43Ta14Ti20Zr7; Cr15Hf6Nb43Ti15Zr21; Cr15Hf6Nb41Ti10Zr28; Cr10Nb49Ta14Ti20Zr7; Cr5Nb47Ta14Ti20Zr14; Nb45Ta14Ti20Zr21; Nb38Ta21Ti20Zr21; Al6Cr5Nb39Ta14Ti15Zr21; Al9Nb36Ta21Ti20Zr14; Al9Cr15Nb34Ta14Zr28; Al9Nb29Ni15Ta14Ti5Zr28; Al3Nb33Ni5Ta21Ti10Zr28; Al3Nb49Ni5Ta14Ti15Zr14; Al6Nb46Ni15Ta14Ti5Zr14; Al4Cr5Nb30Ta1Ti10V50; Al4Cr5Nb30Ta1Ti20V40; Al2Cr10Ta18Ti20V50; Al8Cr5Ta17Ti20V50; Al8Nb30Ta2Ti20V40; Al4Ni8Ti44V28W16; Al2Nb24Ni8Ti22V44; Al6Ni8Ti26V44W16; Al6Nb24Ni8Ti10V44W8; Al4Cr1Nb30Ni5Ti4V56; Al6Nb16Ni8Ti26V36W8; Al6Cr6Ni8Ti28V36W16; Al2Nb16Ni8Ti14V44W16; Al2Mo8Nb24Ni8Ti22V36; Al2Cr12Nb16Ni8Ti10V44W8; or Al2Hf8Nb24Ni8Ti14V36W8.

Embodiments can include a method for predicting thermodynamic phase of a material. The method can involve obtaining a binary phase diagram for each material to be used as a component of a high-entropy alloy (HEA). The method can involve generating a primary feature that is representative of a probability that the HEA will exhibit a solid solution phase and/or an intermetallic phase. The method can involve generating an adaptive feature that is representative of a factor favoring formation of a desired intermetallic HEA phase. The method can involve encoding the primary feature and/or the adaptive feature with thermodynamic data associated with formation of HEA alloy phases. The method can involve generate an output representation of the HEA alloy phases for a material under analysis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the present disclosure will become more apparent upon reading the following detailed description in conjunction with the accompanying drawings, wherein like elements are designated by like numerals, and wherein:

FIG. 1 shows an exemplary flow diagram for predicting thermodynamic phase of a material;

FIG. 2 shows an exemplary flowchart of evolution of an exemplary alloy design framework based on a set of primary features and adaptive features;

FIG. 3 are three-dimensional pots showing well-defined HEA phase regions at T>0.7 Tₘ in various 3D representations of feature space;

FIG. 4 shows a demonstration of the binary phase field percentage calculation;

FIG. 5 shows binary phase diagrams that can be used to determine the binary phase separation percentage for HEA Al2CoCrCuNi, wherein (a) Cr—Cu shows a complete phase separation effect, and (b) shows an overlay of the Co—Cu phase diagram to illustrate a method to determine the phase separation parameter;

FIG. 6 shows a plot of machine learning prediction success rates for different phases of HEAs;

FIG. 7 shows a crystal structure of the X₂YZ Heusler phase—Symbols: X(red), Y(green), and Z(blue);

FIG. 8 shows visualizations of the partitioning of HEA_{L21} and HEA_{non-L21} phase regions using adaptive features;

FIG. 9 shows visualizations of the partitioning of HEAB2 and HEAnon-B2 phase regions using the adaptive features;

FIG. 10 shows an exemplary flow chart of an implementation of feature engineering in Heusler phase prediction;

FIG. 11 shows crystal structures of a hypothetical high-entropy intermetallic compound based on A4B4 and its two sublattices A and B;

FIG. 12 shows candidate machine learning features and their roles in synthesizability and physical properties; and

FIG. 13 is a flowcharts representing the flow of processes in the two modules called “Machine Learning Model Processes” and “Materials Design Processes”, respectively.

### DETAILED DESCRIPTION

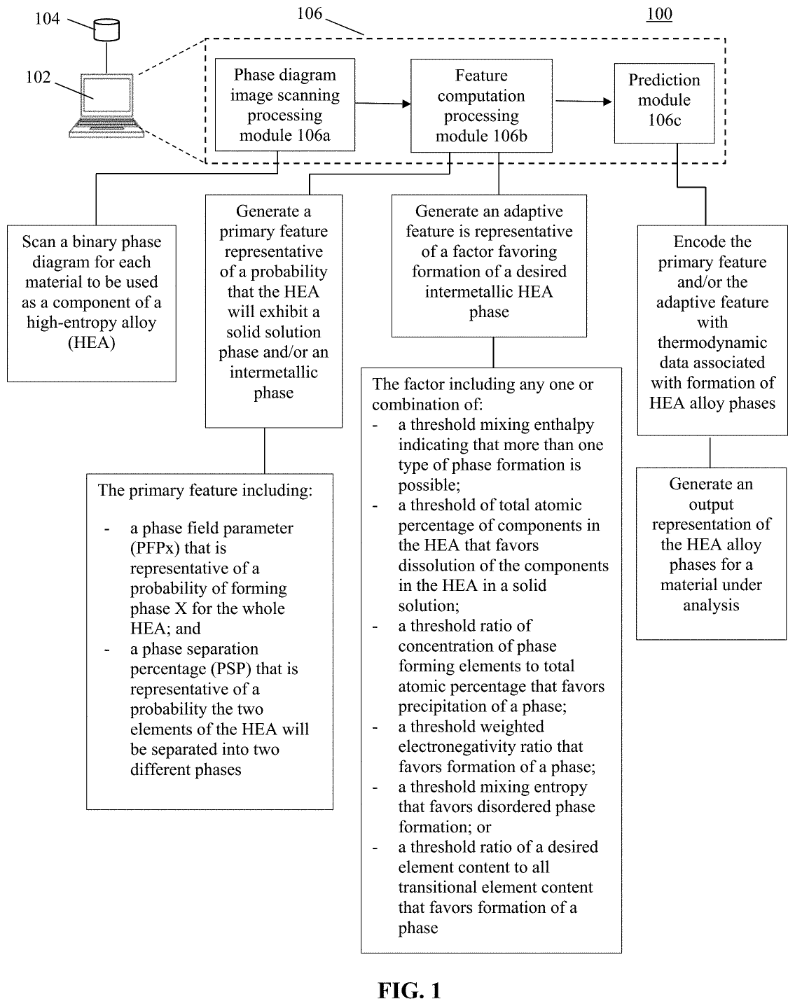

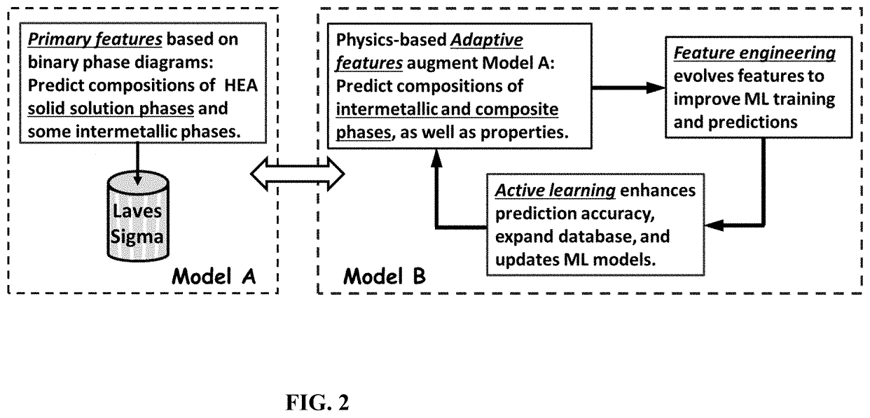

Referring to FIGS. 1-2, embodiments can relate to a system 100 for predicting thermodynamic phase of a material. As will be explained herein, the disclosed systems 100 and methods involve use of a model for predicting thermodynamic phase of a material. The model can be thought of as a synergistic utilization of two separate models. The first model can be referred to herein as Model A. The second model can be referred to herein as Model B. Model A's primary function is to generate primary features (to be explained later) to be used as a predictor of thermodynamic phase of a material, whereas Model B's primary function is to generate adaptive features (to be explained later) as a predictor for thermodynamic phase of a material.

In a recent publication²⁵, the inventors described a novel alloy design approach based on the use of phenomenological features formulated from constituent binary phase diagrams. This machine learning model (Model A) achieves high accuracy in accounting for the compositions of nearly 1,000 HEAs, particularly regarding the solid-solution phases (SS). Model A has been validated experimentally. Building on the machine learning (ML) of Model A, the inventors have developed additional ML models, collectively called Model B, that utilizes adaptive features inspired by physics and experiments to explore the vast and untapped potential of HEA alloys beyond the SS phases. This lead to the formation of new HEAs. The new HEAs, which include intermetallic phases (IMs) and composites composing SS phases and IMs, can be designed for outstanding structural and functional properties. Thus, embodiments disclosed herein relate to the synergistic utilization of the ML Model A and Model B to efficiently explore the complex compositional landscape of multi-component alloys in order to design the new HEAs.

Model A pioneers the use of phenomenological features (descriptors) built on ˜4,700 widely accessible binary alloy phase diagrams, replacing conventional empirical features. Phase diagrams manifest the thermodynamic state of elemental mixtures. The rich information encoded therein can be exploited in a combinatorial manner to project phase formation in multi-component alloys. These phenomenological features are referred to herein as primary features. The use of phenomenological features enhances the efficacy of ML in predicting the formation of specific HEA phases, starting with those that exhibit solid-solution regions in the phase diagrams. The phenomenological ML model predicts SS and limited IM phases in the complex composition space, where SS include A1 (FCC), A2 (BCC), and A3 (hexagonal) phases, and IM phases are principally the Al—(Ni, Fe, Co) type B2 phases and Laves, and Sigma phases.

Model B can be used to design a broad class of intermetallic phases such as ordered BCC (B2), Heusler, half-Heusler, and ordered FCC (L12) phases. Most of these IM phases are not found in the binary alloy phase diagrams, and therefore cannot be predicted by only using Model A. Prospective HEA phases are first examined using Model A for the potential formation of composites or IMs. The synergistic use of Model A and Model B involves human intervention that helps to minimize the number of experiments. Model B incorporates adaptive features constructed for specific IM phases of interest. With known methods, the traditional approach employs features expressed in terms of single or combination of chemistry and physics-based parameters, e.g., atomistic parameters such as atomic radius, electron configuration, and melting point; chemical parameters such as electronegativity, valence state, and stoichiometry; and thermal and physical property parameters such as formation enthalpy, elastic modulus, electrical conductivity, thermal expansion coefficient, Seebeck coefficient, and magnetization. In contrast, the inventive method creates specific features adapted to specific intermetallic phases as necessitated by the different sets of factors governing the formation of these different phases.

A schematic of the inventive alloy design can be appreciated from FIG. 2. FIG. 2 shows a flowchart illustrating the evolution of the alloy design framework foundationed on a set of primary features and adaptive features. Examples of predicted solid solution phases and intermetallic phases are listed above and specific intermetallic phases will be discussed later. Note that “feature” and “descriptor” can be used interchangeably.

As will be explained in more detail, the systems 100 and methods disclosed herein can be enhanced by feature engineering²⁶ that evolves the initial features to optimize outcomes through sequential training. The inventive method can be further enhanced in prediction accuracy by using active learning²⁷ through the interaction of ML with experiments to update features and train ML algorithms. The learning method can be employed to expand the database outside existing compositional ranges to enable discovery besides alloy optimization.

The inventive methods is founded on the synergistic deployment of phenomenological features and adaptive features, providing a framework to accelerate the design of complex composition alloys, specifically high-entropy solid solution alloys and composites as well as intermetallic compounds for outstanding structural and functional properties, such as mechanical, thermal, magnetic, and thermoelectric properties to name a few. The inventive methods can provide efficient optimization of broad classes of complex composition alloys, efficient discovery of broad classes of complex composition alloys, and can achieve much-improved prediction accuracies compared with other methods in identifying specific phases, such as solid solutions, intermetallic compounds, and composites.

The inventive methods for alloy design represent a significantly different approach from prior art. For instance:
A large number of widely accessible binary alloy phase diagrams, potentially up to ˜4,700, are directly utilized in data-driven alloy design for the first time. Phase diagrams provide useful elemental mixing information that can be exploited to provide indiscriminate high-entropy alloys selection feasibility.
The primary features computed from phase diagrams are encoded with thermodynamics data associated with the formation of alloy phases. Thus, in comparison with the prior art, the primary features can better represent experimental data, enabling machine learning to produce more accurate predictions of HEA solid solution A1, A2, and A3 phases, as well as intermetallic B2, Laves, and Sigma phases.
Since A1, A2, A3, and intermetallic B2, Laves, and Sigma phases constitute the dominant high-entropy phases, the high prediction accuracy obtained (represented by success percentages) provides a robust framework for the synergistic application of Model A and Model B to explore HEA composites and intermetallics.
The use of adaptive features tailored towards each individual type of IM phase provides a rapid-throughput adaptable method for the accurate prediction of broad classes of high-entropy intermetallic compounds and composites.
The efficient prediction of high-entropy alloy compositions can be automated with machine learning optimization to provide a robust framework in achieving superior materials properties.
The machine learning enabled high-throughput (HTP) screening of HEAs has the advantage of not being dependent on the availability and depth of thermodynamical databases (e.g., Calculation of Phase Diagram, CALPHAD) and the demand of computational resources (e.g., first-principle calculations). The HTP screening can rapidly down select HEAs for detailed study by CALPHAD and first-principle calculations.
The inventive models can be integrated with popular adaptive design strategy (aka active learning) to enhance further the prediction accuracy of HEA phase formation and properties
The inventive alloy design framework accelerates materials screening for more in-depth scientific study and technological development while optimizing the use of computational time.
For demonstration, the structural phases of several dozen alloys randomly selected outside the current high-entropy alloy compositional regions are predicted. The overall prediction success rate for specific phases is near 85-90%, significantly exceeding those reported by other groups. Prior prediction methods with comparable database and success rates can only predict HEA broad phase categories but not individual specific phases.

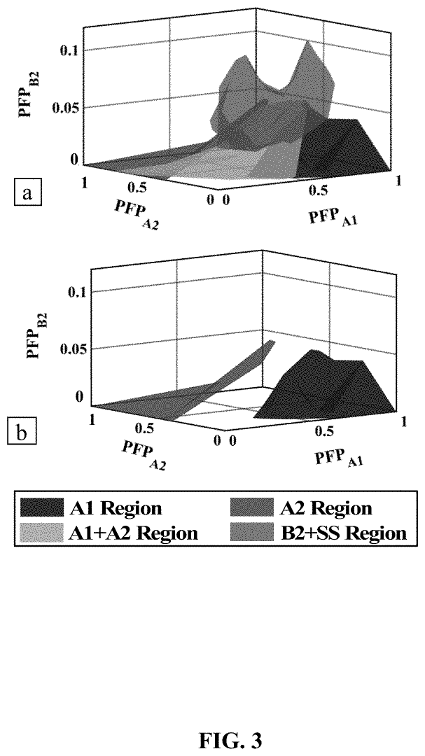

FIG. 3 shows plots illustrating well-defined HEA phase regions at T>0.7Tₘ in various 3D representations of the feature space. Phases A1: FCC, A2: BCC, B2: ordered BCC, SS: solid solution. The axes labels denote features. The effectiveness of the alloy design platform based on Model A is evident in that the temperature region of interest, defined by T>0.7Tₘ (Tₘ is the melting point of the alloy), is usually where the alloys are processed and manufactured. The binary phase diagrams are used to construct a set of primary features that define a high-dimensional feature (descriptor) space. Using the primary features constructed, the current ˜1,000 HEA phases are found to be partitioned into well-defined regions in the feature space with an overall accuracy reaching 85%²⁵. As shown in FIG. 3, the partitioned regions in two three-dimensional (3D) representations of the seven-dimensional (7D) feature space are illustrated. The feature space has direct connections to the compositional space, which enables alloy design. The majority of the current ˜1,000 high-entropy alloys are solid solution alloys consisting of single phase or mixtures (as in a composite) of the A1 (face-centered cubic FCC), A2 (body-centered cubic BCC), A3 (hexagonal close-packed HCP), and B2 (CsCl structure, ordered BCC) structures. For validation, ˜50 randomly selected new compositions were evaluated. The prediction success rate was about 83%.

The efficacy of the alloy design is further evident in the prediction of high-entropy alloy composite formation and improved material properties by deploying Model B. Including adaptive features in stage (ii), the prediction accuracy of intermetallic compounds formation is achieved with near 90% accuracy. One example is the Heusler compound (L2₁ structure) with general composition X₂YZ, e.g., Ni₂TiAl. The Heusler phase has a superior creep resistance that resulted in the superior mechanical properties of some reported high-entropy alloy composites²⁸. Other intermetallic phases, such as those with L1₂ (Cu₃Au) structure, can also be considered. Feature engineering and active learning are integrated within the ML models to provide a universal framework for exploiting the balance of desirable properties inherent to the individual phases in HEAs and expanding the dataset.

The system 100 can include a processor 102 in operative association with memory 104. Any of the processors 102 discussed herein can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors 102 herein includes Graphics Processing Units (GPUs), Field Programmable Gate Arrays (FPGAs), Central Processing Units (CPUs), etc. Any of the memory 104 discussed herein can be computer readable memory configured to store data. The memory 104 can include a volatile or non-volatile, transitory or non-transitory memory (e.g., as a Random Access Memory (RAM)), and be embodied as an in-memory, an active memory, a cloud memory, etc. Embodiments of the memory 104 can include a processor module and other circuitry to allow for the transfer of data to and from the memory 104, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The processor 102 can include plural processing modules 106. The processing module 106 can be embodied as software and stored in memory 104, the memory 104 being operatively associated with the processor 102. In some embodiments, the processing module 106 can be embodied as a web application, a desktop application, a console application, etc.

The plural processing modules 106 can include a phase diagram image scanning processing module 106a configured to scan a binary phase diagram for each material to be used as a component of a high-entropy alloy (HEA). The phase diagram image scanning processing module 106a can include or be in operative association with a camera or other imaging device. Binary phase diagrams of materials to be used as components of the HEA can be pulled from a data source (e.g., a database). The data source can be part of the system 100 (e.g., can be part of the memory 104) or be in operative communication with the system 100. The data source can include a library of binary phase diagrams that are catalogued for easy identification and retrieval. When a material is selected for use as a component, or potential component, of a HEA, the processor 102 can cause the phase diagram image scanning processing module 106a to pull a binary phase diagram for that material and scan it. The phase diagram image scanning processing module 106a can include image processing algorithms that utilize object identification and processing techniques, such as Gabor filtering, for example, to facilitate feature identification within the phase diagrams.

The plural processing modules 106 can include a feature computation processing module 106b configured to generate a primary feature and an adaptive feature. The primary feature is representative of a probability that the HEA will exhibit a solid solution phase and/or an intermetallic phase. The primary feature includes a phase field parameter (PFPₓ) that is representative of a probability of forming phase X for the whole HEA. The primary feature also includes a phase separation percentage (PSP) that is representative of a probability that two elements of the HEA will be separated into two different phases.

The adaptive feature is representative of a factor favoring formation of a desired intermetallic HEA phase. The factor can include any one or combination of: a threshold mixing enthalpy indicating that more than one type of phase formation is possible; a threshold of total atomic percentage of components in the HEA that favors dissolution of the components in the HEA in a solid solution; a threshold ratio of concentration of phase forming elements to total atomic percentage that favors precipitation of a phase; a threshold weighted electronegativity ratio that favors formation of a phase; a threshold mixing entropy that favors disordered phase formation; or a threshold ratio of a desired element content to all transitional element content that favors formation of a phase.

The plural processing module 106 can include a prediction module 106c configured to encode the primary feature and/or the adaptive feature with thermodynamic data associated with formation of HEA alloy phases to provide an output representation of the HEA alloy phases for a material under analysis. Thermodynamic data for a given material is well documented and widely accessible (e.g., via JANAF tables). Thermodynamic data can include entropy, enthalpy, Gibbs free energy, heat capacity, etc. The thermodynamic data can be pulled from the same or different data source used to pull the binary phase diagrams. This data can be placed in a virtual array to generate a virtual table. The primary feature(s) and/or the adaptive feature(s) can be tabulated along with other thermodynamic data about a specific material, thereby encoding the primary feature(s) and/or the adaptive feature(s) with the thermodynamic data.

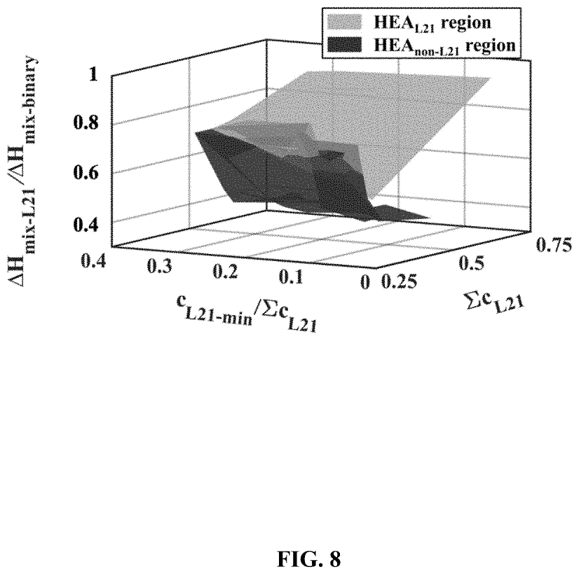

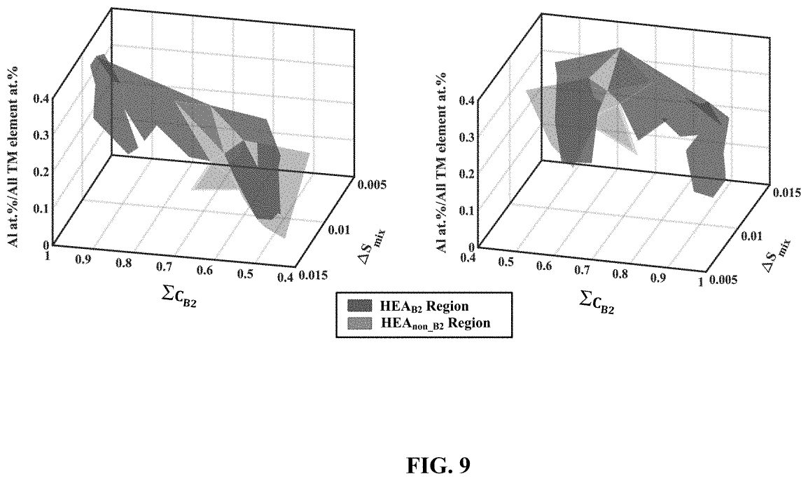

Referring to FIGS. 8-9, the prediction module 106c can be configured to generate as the output a compositional space plot for the HEA alloy phases. For instance, the prediction module 106c can use the encoded thermodynamic data to develop compositional space plots for material used or to be used for the HEA. As noted above, the encoded thermodynamic data includes the probability that the HEA will exhibit a solid solution phase and/or an intermetallic phase under certain conditions. The encoded thermodynamic data also includes factors favoring formation of a desired intermetallic HEA phase. Thus, the compositional space plot can be a representation of the HEA alloy phases that will be formed using the desired materials.

Details of how algorithms that may be used for governing operation of the feature computation processing module 106b and the prediction module 106c are discussed next.

The feature computation processing module 106b can be configured to define a temperature-composition region for the primary feature that is a region on a binary phase diagram bounded by a melting temperature Tₘ and a phase formation temperature T_{pf}. For instance, primary features can be constructed by using the temperature-composition regions in the binary alloy phase diagrams. The regions can be defined to be bounded by the melting temperature T. and phase formation temperature T_{pf}. The processing annealing temperature of the alloys lies above T_{pf}. Tₘ can be determined from the binary phase diagrams with the following equation:

$${T}_{m} = \frac{{\sum }_{i \neq j} {T}_{i - j} \times {c}_{i} \times {c}_{j}}{{\sum }_{i \neq j} {c}_{i} \times {c}_{j}} \qquad ( Eqn . 1 )$$

where Tᵢ₋ⱼ is the binary liquidus temperatures on the binary phase diagram of i-j elements when a relative ratio of two elements of the binary phase diagram is cᵢ:cⱼ.

The phase formation temperature (T_{pf}) is the temperature where rapid phase evolution ceases. T_{pf} is approximated to be T_{pf}≈0.8 Tₘ, where undercooling usually ceases. The postproduction annealing usually occurs near or slightly above T_{pf}. It should be noted that this is a very high T, which leads to high thermal stability for HEAs being designed by the inventive method. As will be demonstrated later, the inventive method can be used to design HEAs with high strength and high ductility, along with high thermal stability. For instance, HEAs can be designed exhibiting 2 Gpa or greater (strengths significantly higher than structural steel) and Poisson's ratios >=0.32 (high ductility). Thus, the inventive method can facilitate designing HEAs with high thermal stability (T_{pf}≈0.8 Tₘ), high strength (>=2 Gpa), and/or high ductility (>=0.32).

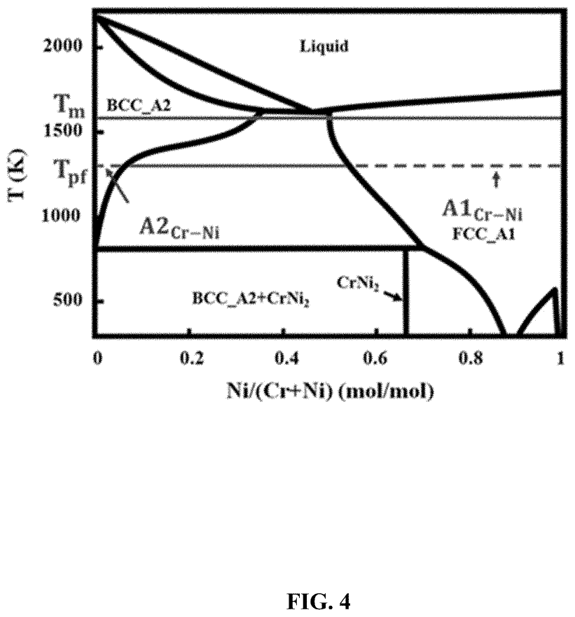

As noted above, the primary feature includes a phase field parameter (PFPₓ) that is representative of a probability of forming phase X for the whole HEA. Explanation of the PFP can begin with an example. HEA Al₂CoCrCuNi has a predicted Tₘ=1569 K. FIG. 4 can be used to demonstrate a binary phase field percentage calculation. In FIG. 4, it is seen that high concentrations of Cr favor BCC formation, while high concentrations of Ni favor FCC formation. Under the assumption of equally sampling all binary configurations, the probability of Cr—Ni favoring BCC formation locally is the binary phase field percentage of the BCC phase. This percentage is the line segment between the two intersection points of an isotherm at T_{pf} and the phase boundary of the BCC phase. In this case, it is approximately 5%, and is denoted as A2_{Cr-Ni}. Similarly, A1_{Cr-Ni}, the probability of favoring FCC formation, is approximately 44%.

The probability of forming phase X locally for i-j elements is the binary phase field percentage of phase X on an i-j phase diagram, and is denoted as Xᵢ₋ⱼ. The probabilities of forming a specific phase from all atomic pairs are integrated for an overall probability. The probability of forming phase X for the whole HEA is the Phase Field Parameter (PFP_{X}), and it is calculated as the weighted average of all constituents Xᵢ₋ⱼ by:

$${PFP}_{X} = \frac{{\sum }_{i \neq j} {X}_{i - j} \times {c}_{i} \times {c}_{j}}{{\sum }_{i \neq j} {c}_{i} \times {c}_{j}} \div 100 \% \qquad ( Eqn . 2 )$$

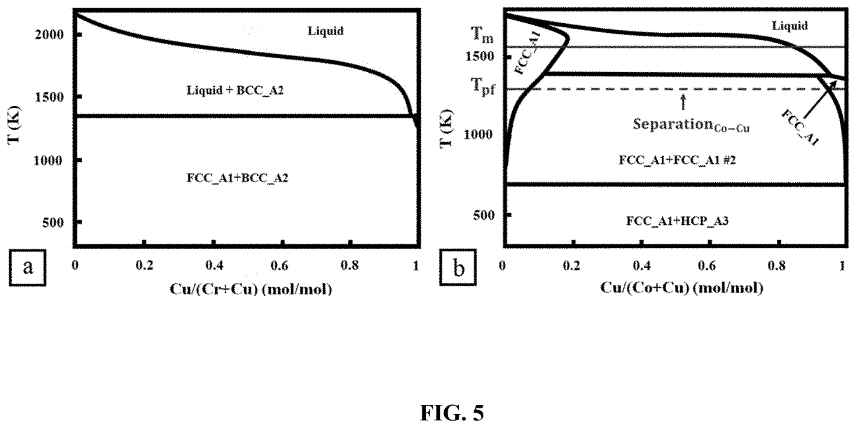

FIG. 5 shows binary phase diagrams used to determine the binary phase separation percentage for HEA Al₂CoCrCuNi: (a) Cr—Cu shows a complete phase separation effect; (b) overlay of the Co—Cu phase diagram illustrating the method to determine the phase separation parameter. A miscibility gap is formed when the interatomic repulsion gives rise to positive heat of mixing ΔHₘᵢₓ, causing phase separation that results in the formation of multiple phases such as FCC+BCC. FIG. 5 shows two phase diagrams with phase separation effects. In FIG. 5, (a) shows that the Cr and Cu tend to stay in different phases; (b) shows that the separation effect still exists due to the positive ΔHₘᵢₓ, although partial elemental mixing can exist marginally at high temperatures.

The phase separation percentage represents the probability of two elements being separated into two different phases. The binary phase separation percentages from all atomic pairs are combined to calculate the Phase Separation Parameter (PSP) of HEA with the following equation:

$$PSP = \frac{{\sum }_{i \neq j} {Separation}_{i - j} \times {c}_{i} \times {c}_{j}}{{\sum }_{i \neq j} {Mixing}_{i - j} \times {c}_{i} \times {c}_{j}} \qquad ( Eqn . 3 )$$

where Separationᵢ₋ⱼ and Mixingᵢ₋ⱼ are the binary phase separation percentage and mixing percentage between i-j pair. The combined total of Separationᵢ₋ⱼ and Mixingᵢ₋ⱼ is 100%. Separationᵢ₋ⱼ=0% if the phase separation is absent from a phase diagram.

The feature computation processing module 106b can be configured to determine a PFPₓ for any one or combination of: PFP_{A1}, which is representative of an A1 (FCC) phase; PFP_{A2}, which is representative of an A2 (BCC) phase; PFP_{B2}, which is representative of an Al—(Ni, Fe, Co) type B2 phase; PFP_{A3}, which is representative of an A3 (hexagonal) phase; PFP_{Laves}, which is representative of a Laves phase; or PFP_{Sigma}, which is representative of a Sigma phase. In some embodiments, the feature computation processing module 106b can be configured to generate the primary feature and/or the adaptive feature using machine learning techniques.

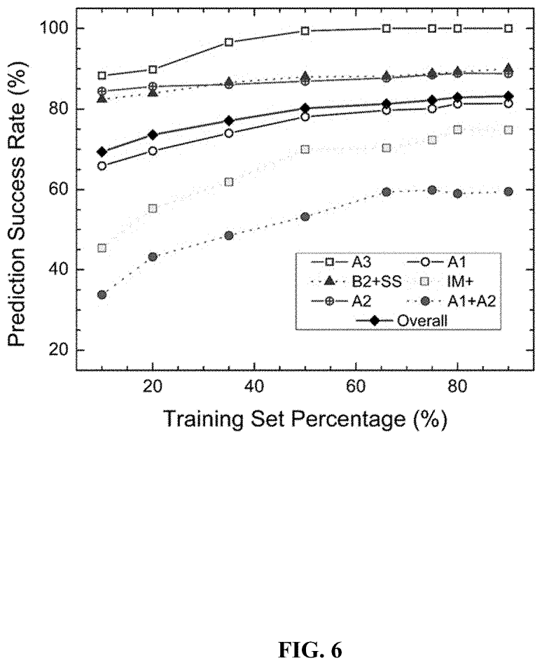

For instance, seven parameters: PFP_{A1}, PFP_{A2}, PFP_{B2}, PFP_{A3}, PFP_{Laves}, PFP_{Sigma}, and PSP can be defined using the methods discussed herein and categorized into seven primary features. ML can be utilized to perform a quantitative analysis of the compositional distribution of phase fields organized in the high-dimensional parameter space. The parameters can be used as features in the ML model, wherein a ML classifier, Random Forest, can be used. The data can be used as the training set and test set, with training set percentages from 10% to 90%. The phase prediction success rates are shown in FIG. 6. The phase categories are A1, A2, A3, A1+A2, B2+SS, and IM+, which denotes a mixture of intermetallic and miscellaneous phases. The overall prediction success rate approaches ˜80-85% for training set percentages of 60-90%. The prediction accuracy is generally high, not only for the single-phase A1, A2, and A3, but also for the HEA composites that contain the ordered B2 phase. The accurate prediction of the B2 phase is crucial as it has shown an effect in improving the mechanical properties.

FIG. 6 shows ML prediction success rates for different phases of HEAs, and Table I shows counts of different HEA phases.

TABLE I
Counts of different HEA phases
Count of HEA [As cast + Annealed]

| Overall | A1 | A2 | A3 | A1 + A2 | B2 + SS | IM+ |
|---|---|---|---|---|---|---|
| 828 | 126 | 178 | 14 | 72 | 290 | 148 |

The predicted composition-phase relationships were validated experimentally. High entropy alloy phases predicted by the invention models were validated experimentally. About four dozen alloy compositions were randomly selected from outside the existing compositional regions. The alloy ingots were prepared by melting mixtures of high-purity (>99.7%) commercial grade elements in an arc furnace with a water-cooled copper hearth under an argon atmosphere. The samples were flipped and melted three more times to ensure homogeneity. The ingots were broken into smaller chunks, and remelted and suction-cast into a copper mold to form 3-mm diameter and 20-mm long rod-shaped samples. Structural investigations were carried out with x-ray diffraction (XRD) analysis using a Cu Kα radiation on a PANalytical Empyrean diffractometer. The alloy phase prediction achieves a success rate near 83%, comparable to that obtained using the test set.

Alloy composites often show improved functional properties besides enhanced mechanical properties compared with single-phase alloys. Composites have a high density of interfaces that can deliver additional functionality. The inventive method can be utilized to predict the formation of intermetallic (IM) phases in HEAs. Different factors influence the formation of different IM phases. Determining what controls the formation of a specific IM phase led to the construction of informed physics-based adaptive features. The application of adaptive features is demonstrated for two IM phases, namely Heusler (L2₁ structure) phase and the ordered BCC (B2 structure) phase.

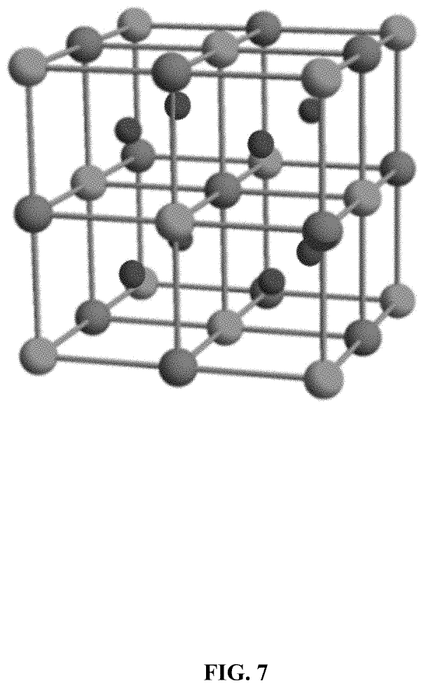

FIG. 7 shows a crystal structure of the X₂YZ Heusler phase, wherein symbols: X(red), Y(green), and Z(blue). Heusler phase (L2₁ structure)—These have the general composition X₂YZ, where the symbols X, Y, and Z are limited to certain elements³⁰. Ni₂TiAl is an example of a Heusler compound that forms as a precipitate phase in the HEA composite. The crystal structure of Ni₂TiAl is shown in FIG. 7. Heusler compounds are of interest to develop HEAs with favorable mechanical properties²⁸. The Heusler phase has a higher creep resistance compared with the B2 phase due to limited slip^{31,32}. Heusler-type Ni—Mn—In—(Co) magnetic shape-memory alloys, which exhibit giant magnetocaloric effect driven by magneto structural transition, are promising refrigeration materials³³. However, the prediction for Heusler phase formation in HEAs is lacking. Four data-based parameters that can influence L21 phase formation are identified as candidate adaptive features:
a) More than one IM phase could form. The mixing enthalpy of the Heusler phase (ΔH_{mix-L21}) can be compared with the most negative binary ΔHₘᵢₓ in the HEA (ΔH_{mix-binary}), which suggests that the ratio ΔH_{mix-L21}/ΔH_{mix-binary} is a reasonable choice as an adaptive feature.
b) The total atomic percentage of X, Y, and Z in the HEA, ΣC_{L21}, can be assumed to infer the tendency of Heusler phase forming. A low ΣC_{L21} could favor the dissolution of X, Y, and Z components in the HEA solid solution. Thus, ΣC_{L21} can be a good feature candidate.
c) If the concentration of one of the three Heusler phase forming elements is low relative to ΣC_{L21}, there will be a stronger tendency to precipitate the Heusler phase due to a decrease in the configurational entropy. Thus, the relative concentration ratio C_{L21-min}/ΣC_{L21}, where C_{L21-min} denotes the lowest concentration among X, Y, and Z, can serve as an adaptive feature.
d) Weighted electronegativity (χ) represents an atom's ability to pull electrons. An unbalanced χ distribution among the atoms may favor the formation of IM over SS. Thus, Weighted Electronegativity

$$( χ ) Ratio = \frac{{C}_{χ M a x} \times {χ}_{M a x}}{{C}_{χ Mim} \times {χ}_{M i n}}$$

can be defined to demonstrate the unbalanced extent of χ distribution among HEA elements.

FIG. 8 is a visualization of the partitioning of HEA_{L21} and HEA_{non-L21} phase regions using the prescribed adaptive features described herein. The current HEA database has about 150 HEAs that contain Heusler phase forming elements, in which 50 HEAs contain the Heusler phase (HEA_{L21}) and 100 HEAs do not contain the Heusler phase (HEA_{non-L21}). The HEAs are annealed to mitigate the effect due to rapid cooling that could circumvent the formation of the Heusler phase. The efficacy of the adaptive features prescribed herein is demonstrated in the successful partitioning of the HEA_{L21} and HEA_{non-L21} phase fields plotted in the 3D feature space, as shown in FIG. 8. ML can be employed to classify the ˜150 HEAs into HEA_{L21} and HEA_{non-L21}. The use of Random Forest as the ML classifier returns moderately high prediction success rates of about 75% and 84% for HEA_{L21} and HEA_{non-L21}, respectively. The results are shown in Table II.

TABLE II
ML training success rates for ~50 HEA_{L21} and ~100 HEA_{no-L21}

| Training | HEA_{L21} Success Rate | HEA_{non-L21} Success Rate |
|---|---|---|
| % | (%) | (%) |
| 90 | 75 | 84 |
| 80 | 73 | 83 |
| 75 | 71 | 83 |
| 66 | 72 | 82 |
| 50 | 71 | 81 |

Ordered-BCC phase (B2 structure)—The formation of the B2 phase in refractory HEA is of interest in that the high strengths of the composites can be retained at high temperatures. Refractory B2 compounds were found with the constitution Al—X—Y, where X═Ti, Zr, and/or Hf, and Y═Cr, Mo, Nb, Ta, V, and/or W.³⁴ The prediction model for B2 formation in the Al-refractory element system is still lacking. Three adaptive ML features were developed to identity its formation capability
1. The total atomic percentage of X, Y elements, and Al in the HEA, ΣC_{B2}, is assumed to infer the tendency of B2 phase forming. The low ΣC_{B2} may infer the dissolution of B2 forming elements in the solid solution. Thus, ΣC_{B2} can be a useful feature candidate.
2. A high value of parameter HEA mixing entropy favors the disordered phase formation. ΔSₘᵢₓ can indicate the ordering level as a ML feature.
3. Al content is crucial in forming the B2 phase. The ratio of Al content to all transition elements content (Al at. %/All TM element at. %) can influence the B2 formation tendency.

FIG. 9 shows visualizations of the partitioning of HEA_{B2} and HEA_{non-B2} phase regions using the prescribed adaptive features described herein. The current HEA database has about 88 HEAs that contain refractory B2 phase forming elements, in which 53 HEAs contain the B2 phase (HEA_{B2}) and 35 HEAs do not contain the B2 phase (HEA_{non-B2}). The partitioning of the HEA_{B2} and HEA_{non-B2} phase fields is plotted in the 3D feature space, as shown in FIG. 9.

ML with Random Forest classifier returns prediction success rates of about 75% and 65% for HEA_{B2} and HEA_{non-B2}, respectively. The results are shown in Table III.

TABLE III
ML training success rates for 53 HEA_{B2} and 35 HEA_{non-B2}.

| Training | HEA_{B2} Success Rate | HEA_{non-B2} Success Rate |
|---|---|---|
| % | (%) | (%) |
| 90 | 75 | 65 |
| 80 | 74 | 65 |
| 75 | 74 | 63 |
| 66 | 74 | 62 |
| 50 | 72 | 59 |

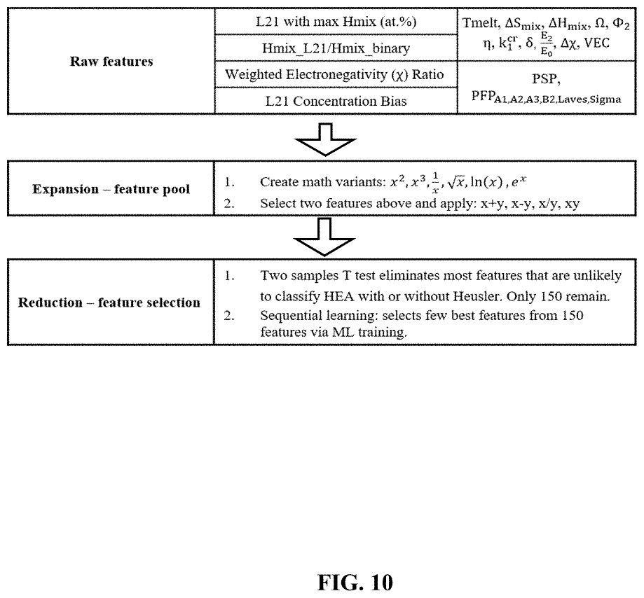

In some embodiments, the feature computation processing module 106b can be configured to optimize the primary feature and/or the adaptive feature via sequential training. FIG. 10 shows a flow chart for feature engineering Heusler phase prediction. Feature engineering can be used to expand the parameter pool by mathematically manipulating the constructed set of ML features to enhance and optimize ML training. Feature engineering can involve a process of extracting features (characteristics, properties, attributes, etc.) from raw data. The features can then be used by predictive models. The over deployment of features in ML can cause overfitting and long computation time. Feature engineering can help to reduce the dimension of feature space by performing various mathematical combinations of the features, while not losing much information. The mathematical expression of each feature can be fine-tuned sequentially to predict the phase formation better. FIG. 10 shows a flow chart demonstrating an exemplary way to use feature engineering in Heusler phase prediction. Starting with 22 initial features, including the four adaptive features²⁹ discussed herein and eighteen features from literature, mathematical variants can be created to expand the feature pool. With over 30,000 engineered features, a two-sample T-test can first reduce the inefficient engineered features. Sequential learning can then be applied to determine the most important features.

Active learning can be employed to exploit small databases. To date, despite the report of ˜1,000 HEAs with diverse compositions and structural phases, the potential number of HEAs remains exponentially larger. Active learning utilizes an iterative process supported by experimentation that gathers new data in the untapped compositional regions, significantly expanding the database, while also sharpening the prediction.

Predictions of new high-entropy alloy phases using ML primary features have been demonstrated by the inventors²⁵. Within the invention design framework, the ML models are further developed to optimize phase formation and materials properties simultaneously. Different applications involve different operating conditions, and thus require specific material properties. For example, some applications may require the materials to have good corrosion and oxidation resistance as well as high strength and damage tolerance, and other applications may require high magnetic entropy, thermopower, or piezoelectric coefficient, etc. The strategies for alloy design and discovery are highlighted below.

The high strength and ductility found in HEAs are usually explained in light of solid-solution strengthening and second-phase formation. The mechanical strengths (τ) of alloys can be inferred from the shear modulus (G), as follows:

τ≈0.05G (Eqn. 4)

where G is estimated from the elemental values weighted by the mole fractions of the elements within the effective medium model.

Ductility and toughness can be inferred from the Poisson's ratio (σ) which is also estimated using the effective medium model. The approximate equation obtained is as follows:

$$σ \approx \frac{\sum \frac{{x}_{i} {σ}_{i}}{1 + {σ}_{i}}}{1 - \sum \frac{{x}_{i} {σ}_{i}}{1 + {σ}_{i}}} , \qquad ( Eqn . 5 )$$

where σᵢ is the Poisson's ratio of the element and xᵢ is the mole fraction. Ductile alloys tend to show σ >0.3.

It can be shown²⁵ that the disclosed ML model can predict single-phase HEAs with the face-centered-cubic (FCC) and body-centered cubic (BCC) structures known as FCC and BCC solid solutions (SS), as well as SS+B2 (ordered BCC) and SS+L2₁ (Heusler) composite phases.

The ML model can be employed to design HEA solid-solutions and composites with specific structural properties. A technological area of high importance demands high-performance structural alloys upon prolonged exposure in extreme environments. This requires the materials to retain high strengths and damage tolerance at high temperatures (>1000° C.). The structural alloys also have high resistance against mechanical stress, thermal stress, and corrosion. One such application involves turbine blades for gas turbines widely used for electric power generation and aircraft propulsion. The gas-phase environment of gas turbines ideally would reach temperatures as high as 1800° C. in order to achieve near-Carnot efficiency. The design of HEAs for meeting the basic requirement must consider high melting temperature (Tₘ) for thermal stability, high elastic moduli for high strength, and higher than critical Poisson ratio σ˜0.3 for ductility and toughness, as well as the use of appropriate elements for passivation.

The inventive ML algorithm facilitates design of HEAs meeting the demanding materials requirements. High strength and ductility can be attained through solid-solution and particle inclusion strengthening, such as through lattice deformation and defect network, and formation of HEA composites that contain B2, L2₁, and other intermetallic phases, respectively. In addition, short-range order (SRO) that exists in HEAs also tends to promote strengthening. SRO exists in HEAs that contain Al, V, Zr, and Hf due to atomic size mismatch and chemical bonding effects. Mechanical strengthening can also be achieved in multiscale hierarchical structures by design. Consideration of corrosion resistance is also given to passivating elements such as Al, Cr, and Mo. The predicted HEAs have Tₘ>1900° C. and Poisson's ratio preferably greater than 0.35 estimated using effective medium models. Only low-density HEAs are selected (below 9 g/cc). The designed HEA systems include BCC, BCC+B2, and BCC+L2₁ phases. Currently, the computer program has scanned more than 10⁶ compositions. The compositions listed in Table IV are the representatives that have passed the properties filters. These HEA alloy systems are designed to have load-bearing strengths, either yield or ultimate fracture strengths around or greater than 2 GPa.

TABLE IV
A list of the alloys with the corresponding Poisson's ratios, Tₘ's,
densities, phases, and strengths predicated by the alloy design framework

| | | | Density | Strength | Poisson's | |
|---|---|---|---|---|---|---|
| | Compositions | Tm(C.) | (g/cc) | (GPa) | ratio | Phase |
| NbV-based | Al3Nb47Ta18Ti20V12 | 2299 | 8.9 | 2.1 | 0.37 | BCC |
| BCC HEA | Al6Nb50Ta12Ti20V6W6 | 2329 | 8.9 | 2.1 | 0.36 | BCC |
| | Al9Nb47Ta12Ti20V6W6 | 2288 | 8.7 | 2.1 | 0.36 | BCC |
| | Al3Nb41Ti20V18W6Zr12 | 2079 | 7.5 | 2.1 | 0.36 | BCC |
| | Nb50Ta12Ti20W6Zr12 | 2288 | 9.0 | 2.1 | 0.36 | BCC |
| | Nb32Ta18Ti20V24Zr6 | 2141 | 8.6 | 2.2 | 0.36 | BCC |
| | Nb32Ti20V24W12Zr12 | 2106 | 8.1 | 2.2 | 0.35 | BCC |
| | Al3Hf6Nb35Ta12Ti20V24 | 2094 | 8.5 | 2.1 | 0.37 | BCC |
| | Al3Nb41Ta12Ti20V18Zr6 | 2149 | 8.1 | 2.1 | 0.37 | BCC |
| | Al3Nb47Ta18Ti20Zr12 | 2276 | 8.8 | 2.0 | 0.36 | BCC |
| NbV-based | Al9Hf6Nb41Ti20V18W6 | 2109 | 7.8 | 2.0 | 0.36 | BCC + B2 |
| BCC + B2 | Al6Nb32Ta18Ti20V24 | 2152 | 8.5 | 2.2 | 0.36 | BCC + B2 |
| HEA | Al6Nb26Ta12Ti20V30Zr6 | 1998 | 7.6 | 2.1 | 0.36 | BCC + B2 |
| | Al3Nb41Ta12Ti20V18Zr6 | 2149 | 8.1 | 2.1 | 0.37 | BCC + B2 |
| | Al6Nb48Ta12Ti10W6Zr18 | 2280 | 8.9 | 2.0 | 0.36 | BCC + B2 |
| | Al9Nb29Ti20V30W6Zr6 | 1985 | 7.0 | 2.1 | 0.36 | BCC + B2 |
| Nb-based | Al3Nb42Ta21Ti20Zr14 | 2275 | 8.9 | 2.1 | 0.36 | BCC |
| BCC HEA | Al6Nb39Ta21Ti20Zr14 | 2246 | 8.8 | 2.0 | 0.36 | BCC |
| | Nb50Ta12Ti20W6Zr12 | 2288 | 9.0 | 2.1 | 0.36 | BCC |
| | Cr5Hf6Nb48Ta7Ti20Zr14 | 2145 | 8.3 | 2.0 | 0.36 | BCC |
| | Cr10Hf6Nb43Ta14Ti20Zr7 | 2199 | 9.0 | 2.2 | 0.35 | BCC |
| | Cr15Hf6Nb43Ti15Zr21 | 2014 | 7.7 | 2.0 | 0.34 | BCC |
| | Cr15Hf6Nb41Ti10Zr28 | 1991 | 7.7 | 2.0 | 0.34 | BCC |
| | Cr10Nb49Ta14Ti20Zr7 | 2222 | 8.6 | 2.2 | 0.35 | BCC |
| | Cr5Nb47Ta14Ti20Zr14 | 2202 | 8.5 | 2.1 | 0.36 | BCC |
| | Nb45Ta14Ti20Zr21 | 2164 | 8.4 | 2.0 | 0.36 | BCC |
| | Nb38Ta21Ti20Zr21 | 2203 | 8.9 | 2.1 | 0.36 | BCC |
| Nb-based | Al6Cr5Nb39Ta14Ti15Zr21 | 2129 | 8.2 | 2.0 | 0.35 | BCC + B2 |
| BCC + B2 | Al9Nb36Ta21Ti20Zr14 | 2209 | 8.6 | 2.0 | 0.36 | BCC + B2 |
| HEA | Al9Cr15Nb34Ta14Zr28 | 1994 | 8.3 | 2.1 | 0.34 | BCC + B2 |
| Nb-based | Al9Nb29Ni15Ta14Ti5Zr28 | 1882 | 8.3 | 2.0 | 0.35 | BCC + L21 |
| BCC + L21 | Al3Nb33Ni5Ta21Ti10Zr28 | 2169 | 9.0 | 2.1 | 0.36 | BCC + L21 |
| HEA | Al3Nb49Ni5Ta14Ti15Zr14 | 2221 | 8.6 | 2.0 | 0.36 | BCC + L21 |
| | Al6Nb46Ni15Ta14Ti5Zr14 | 2128 | 8.8 | 2.1 | 0.36 | BCC + L21 |
| V-based | Al4Cr5Nb30Ta1Ti10V50 | 1935 | 6.8 | 2.2 | 0.36 | BCC |
| BCC HEA | Al4Cr5Nb30Ta1Ti20V40 | 1912 | 6.6 | 2.2 | 0.36 | BCC |
| | Al2Cr10Ta18Ti20V50 | 1924 | 8.0 | 2.6 | 0.34 | BCC |
| | Al8Cr5Ta17Ti20V50 | 1913 | 7.6 | 2.4 | 0.34 | BCC |
| | Al8Nb30Ta2Ti20V40 | 1910 | 6.5 | 2.1 | 0.37 | BCC |
| V-based | Al4Ni8Ti44V28W16 | 1965 | 7.5 | 2.6 | 0.33 | BCC + L21 |
| BCC + L21 | Al2Nb24Ni8Ti22V44 | 1801 | 6.5 | 2.2 | 0.36 | BCC + L21 |
| HEA | Al6Ni8Ti26V44W16 | 1983 | 8.9 | 2.5 | 0.34 | BCC + L21 |
| | Al6Nb24Ni8Ti10V44W8 | 1987 | 8.6 | 2.3 | 0.36 | BCC + L21 |
| | Al4Cr1Nb30Ni5Ti4V56 | 1929 | 7.6 | 2.2 | 0.37 | BCC + L21 |
| | Al6Nb16Ni8Ti26V36W8 | 1861 | 8.1 | 2.3 | 0.35 | BCC + L21 |
| | Al6Cr6Ni8Ti28V36W16 | 1981 | 8.9 | 2.6 | 0.32 | BCC + L21 |
| | Al2Nb16Ni8Ti14V44W16 | 2082 | 8.6 | 2.6 | 0.35 | BCC + L21 |
| | Al2Mo8Nb24Ni8Ti22V36 | 1871 | 6.8 | 2.0 | 0.36 | BCC + L21 |
| | Al2Cr12Nb16Ni8Ti10V44W8 | 1921 | 7.7 | 2.6 | 0.34 | BCC + L21 |
| | Al2Hf8Nb24Ni8Ti14V36W8 | 1962 | 8.5 | 2.3 | 0.36 | BCC + L21 |

Thus, in an exemplary embodiment, a high-entropy alloy can be any one or combination of: Al3Nb47Ta18Ti20V12; Al6Nb50Ta12Ti20V6W6; Al9Nb47Ta12Ti20V6W6; Al3Nb41Ti20V18W6Zr12; Nb50Ta12Ti20W6Zr12; Nb32Ta18Ti20V24Zr6; Nb32Ti20V24W12Zr12; Al3Hf6Nb35Ta12Ti20V24; Al3Nb41Ta12Ti20V18Zr6; Al3Nb47Ta18Ti20Zr12; Al9Hf6Nb41Ti20V18W6; Al6Nb32Ta18Ti20V24; Al6Nb26Ta12Ti20V30Zr6; Al3Nb41Ta12Ti20V18Zr6; Al6Nb48Ta12Ti10W6Zr18; Al9Nb29Ti20V30W6Zr6; Al3Nb42Ta21Ti20Zr14; Al6Nb39Ta21Ti20Zr14; Nb50Ta12Ti20W6Zr12; Cr5Hf6Nb48Ta7Ti20Zr14; Cr10Hf6Nb43Ta14Ti20Zr7; Cr15Hf6Nb43Ti15Zr21; Cr15Hf6Nb41Ti10Zr28; Cr10Nb49Ta14Ti20Zr7; Cr5Nb47Ta14Ti20Zr14; Nb45Ta14Ti20Zr21; Nb38Ta21Ti20Zr21; Al6Cr5Nb39Ta14Ti15Zr21; Al9Nb36Ta21Ti20Zr14; Al9Cr15Nb34Ta14Zr28; Al9Nb29Ni15Ta14Ti5Zr28; Al3Nb33Ni5Ta21Ti10Zr28; Al3Nb49Ni5Ta14Ti15Zr14; Al6Nb46Ni15Ta14Ti5Zr14; Al4Cr5Nb30Ta1Ti10V50; Al4Cr5Nb30Ta1Ti20V40; Al2Cr10Ta18Ti20V50; Al8Cr5Ta17Ti20V50; Al8Nb30Ta2Ti20V40; Al4Ni8Ti44V28W16; Al2Nb24Ni8Ti22V44; Al6Ni8Ti26V44W16; Al6Nb24Ni8Ti10V44W8; Al4Cr1Nb30Ni5Ti4V56; Al6Nb16Ni8Ti26V36W8; Al6Cr6Ni8Ti28V36W16; Al2Nb16Ni8Ti14V44W16; Al2Mo8Nb24Ni8Ti22V36; Al2Cr12Nb16Ni8Ti10V44W8; or Al2Hf8Nb24Ni8Ti14V36W8.

In some embodiments, Al3Nb47Ta18Ti20V12 has a BBC phase; Al6Nb50Ta12Ti20V6W6 has a BBC phase; Al9Nb47Ta12Ti20V6W6 has a BBC phase; Al3Nb41Ti20V18W6Zr12 has a BBC phase; Nb50Ta12Ti20W6Zr12 has a BBC phase; Nb32Ta18Ti20V24Zr6 has a BBC phase; Nb32Ti20V24W12Zr12 has a BBC phase; Al3Hf6Nb35Ta12Ti20V24 has a BBC phase; Al3Nb41Ta12Ti20V18Zr6 has a BBC phase; Al3Nb47Ta18Ti20Zr12 has a BBC phase; Al9Hf6Nb41Ti20V18W6 has a BBC+B2 phase; Al6Nb32Ta18Ti20V24 has a BBC+B2 phase; Al6Nb26Ta12Ti20V30Zr6 has a BBC+B2 phase; Al3Nb41Ta12Ti20V18Zr6 has a BBC+B2 phase; Al6Nb48Ta12Ti10W6Zr18 has a BBC+B2 phase; Al9Nb29Ti20V30W6Zr6 has a BBC+B2 phase; Al3Nb42Ta21Ti20Zr14 has a BBC phase; Al6Nb39Ta21Ti20Zr14 has a BBC phase; Nb50Ta12Ti20W6Zr12 has a BBC phase; Cr5Hf6Nb48Ta7Ti20Zr14 has a BBC phase; Cr10Hf6Nb43Ta14Ti20Zr7 has a BBC phase; Cr15Hf6Nb43Ti15Zr21 has a BBC phase; Cr15Hf6Nb41Ti10Zr28 has a BBC phase; Cr10Nb49Ta14Ti20Zr7 has a BBC phase; Cr5Nb47Ta14Ti20Zr14 has a BBC phase; Nb45Ta14Ti20Zr21 has a BBC phase; Nb38Ta21Ti20Zr21 has a BBC phase; Al6Cr5Nb39Ta14Ti15Zr21 has a BBC+B2 phase; Al9Nb36Ta21Ti20Zr14 has a BBC+B2 phase; Al9Cr15Nb34Ta14Zr28 has a BBC+B2 phase; Al9Nb29Ni15Ta14Ti5Zr28 has a BBC+L21 phase; Al3Nb33Ni5Ta21Ti10Zr28 has a BBC+L21 phase; Al3Nb49Ni5Ta14Ti15Zr14 has a BBC+L21 phase; Al6Nb46Ni15Ta14Ti5Zr14 has a BBC+L21 phase; Al4Cr5Nb30Ta1Ti10V50 has a BBC phase; Al4Cr5Nb30Ta1Ti20V40 has a BBC phase; Al2Cr10Ta18Ti20V50 has a BBC phase; Al8Cr5Ta17Ti20V50 has a BBC phase; Al8Nb30Ta2Ti20V40 has a BBC phase; Al4Ni8Ti44V28W16 has a BBC+L21 phase; Al2Nb24Ni8Ti22V44 has a BBC+L21 phase; Al6Ni8Ti26V44W16 has a BBC+L21 phase; Al6Nb24Ni8Ti10V44W8 has a BBC+L21 phase; Al4Cr1Nb30Ni5Ti4V56 has a BBC+L21 phase; Al6Nb16Ni8Ti26V36W8 has a BBC+L21 phase; Al6Cr6Ni8Ti28V36W16 has a BBC+L21 phase; Al2Nb16Ni8Ti14V44W16 has a BBC+L21 phase; Al2Mo8Nb24Ni8Ti22V36 has a BBC+L21 phase; Al2Cr12Nb16Ni8Ti10V44W8 has a BBC+L21 phase; and Al2Hf8Nb24Ni8Ti14V36W8 has a BBC+L21 phase.

In some embodiments the high-entropy alloy can be designed for high thermal stability, ductility, and high strengths. For instance, Al3Nb47Ta18Ti20V12 can have a melting temperature of 2299° C., a density of 8.9 g/cc, a strength of 2.1 GPa, and Poisson's ratio of 0.37. Al6Nb50Ta12Ti20V6W6 can have a melting temperature of 2329° C., a density of 8.9 g/cc, a strength of 2.1 GPa, and Poisson's ratio of 0.36. Al9Nb47Ta12Ti20V6W6 can have a melting temperature of 2288° C., a density of 8.7 g/cc, a strength of 2.1 GPa, and Poisson's ratio of 0.36. Al3Nb41Ti20V18W6Zr12 can have a melting temperature of 2079° C., a density of 7.5 g/cc, a strength of 2.1 GPa, and Poisson's ratio of 0.36. Nb50Ta12Ti20W6Zr12 can have a melting temperature of 2288° C., a density of 9.0 g/cc, a strength of 2.1 GPa, and Poisson's ratio of 0.36. Nb32Ta18Ti20V24Zr6 can have a melting temperature of 2141° C., a density of 8.6 g/cc, a strength of 2.2 GPa, and Poisson's ratio of 0.36. Nb32Ti20V24W12Zr12 can have a melting temperature of 2106° C., a density of 8.1 g/cc, a strength of 2.2 GPa, and Poisson's ratio of 0.35. Al3Hf6Nb35Ta12Ti20V24 can have a melting temperature of 2094° C., a density of 8.5 g/cc, a strength of 2.1 GPa, and Poisson's ratio of 0.37. Al3Nb41Ta12Ti20V18Zr6 can have a melting temperature of 2149° C., a density of 8.1 g/cc, a strength of 2.1 GPa, and Poisson's ratio of 0.37. Al3Nb47Ta18Ti20Zr12 can have a melting temperature of 2276° C., a density of 8.8 g/cc, a strength of 2.0 GPa, and Poisson's ratio of 0.36. Al9Hf6Nb41Ti20V18W6 can have a melting temperature of 2109° C., a density of 7.8 g/cc, a strength of 2.0 GPa, and Poisson's ratio of 0.36. Al6Nb32Ta18Ti20V24 can have a melting temperature of 2152° C., a density of 8.5 g/cc, a strength of 2.2 GPa, and Poisson's ratio of 0.36. Al6Nb26Ta12Ti20V30Zr6 can have a melting temperature of 1998° C., a density of 7.6 g/cc, a strength of 2.1 GPa, and Poisson's ratio of 0.36. Al3Nb41Ta12Ti20V18Zr6 can have a melting temperature of 2149° C., a density of 8.1 g/cc, a strength of 2.1 GPa, and Poisson's ratio of 0.37. Al6Nb48Ta12Ti10W6Zr18 can have a melting temperature of 2280° C., a density of 8.9 g/cc, a strength of 2.0 GPa, and Poisson's ratio of 0.36. Al9Nb29Ti20V30W6Zr6 can have a melting temperature of 1985° C., a density of 7.0 g/cc, a strength of 2.1 GPa, and Poisson's ratio of 0.36. Al3Nb42Ta21Ti20Zr14 can have a melting temperature of 2275° C., a density of 8.9 g/cc, a strength of 2.1 GPa, and Poisson's ratio of 0.36. Al6Nb39Ta21Ti20Zr14 can have a melting temperature of 2246° C., a density of 8.8 g/cc, a strength of 2.0 GPa, and Poisson's ratio of 0.36. Nb50Ta12Ti20W6Zr12 can have a melting temperature of 2288° C., a density of 9.0 g/cc, a strength of 2.1 GPa, and Poisson's ratio of 0.36. Cr5Hf6Nb48Ta7Ti20Zr14 can have a melting temperature of 2145° C., a density of 8.3 g/cc, a strength of 2.0 GPa, and Poisson's ratio of 0.36. Cr10Hf6Nb43Ta14Ti20Zr7 can have a melting temperature of 2199° C., a density of 9.0 g/cc, a strength of 2.2 GPa, and Poisson's ratio of 0.35. Cr15Hf6Nb43Ti15Zr21 can have a melting temperature of 2014° C., a density of 7.7 g/cc, a strength of 2.0 GPa, and Poisson's ratio of 0.34. Cr15Hf6Nb41Ti10Zr28 can have a melting temperature of 1991° C., a density of 7.7 g/cc, a strength of 2.0 GPa, and Poisson's ratio of 0.34. Cr10Nb49Ta14Ti20Zr7 can have a melting temperature of 2222° C., a density of 8.6 g/cc, a strength of 2.2 GPa, and Poisson's ratio of 0.35. Cr5Nb47Ta14Ti20Zr14 can have a melting temperature of 2202° C., a density of 8.5 g/cc, a strength of 2.1 GPa, and Poisson's ratio of 0.36. Nb45Ta14Ti20Zr21 can have a melting temperature of 2164° C., a density of 8.4 g/cc, a strength of 2.0 GPa, and Poisson's ratio of 0.36. Nb38Ta21Ti20Zr21 can have a melting temperature of 2203° C., a density of 8.9 g/cc, a strength of 2.1 GPa, and Poisson's ratio of 0.36. Al6Cr5Nb39Ta14Ti15Zr21 can have a melting temperature of 2129° C., a density of 8.2 g/cc, a strength of 2.0 GPa, and Poisson's ratio of 0.35. Al9Nb36Ta21Ti20Zr14 can have a melting temperature of 2209° C., a density of 8.6 g/cc, a strength of 2.0 GPa, and Poisson's ratio of 0.36. Al9Cr15Nb34Ta14Zr28 can have a melting temperature of 1994° C., a density of 8.3 g/cc, a strength of 2.1 GPa, and Poisson's ratio of 0.34. Al9Nb29Ni15Ta14Ti5Zr28 can have a melting temperature of 1882° C., a density of 8.3 g/cc, a strength of 2.0 GPa, and Poisson's ratio of 0.35. Al3Nb33Ni5Ta21Ti10Zr28 can have a melting temperature of 2169° C., a density of 9.0 g/cc, a strength of 2.1 GPa, and Poisson's ratio of 0.36. Al3Nb49Ni5Ta14Ti15Zr14 has a melting temperature of 2221° C., a density of 8.6 g/cc, a strength of 2.0 GPa, and Poisson's ratio of 0.36. Al6Nb46Ni15Ta14Ti5Zr14 can have a melting temperature of 2128° C., a density of 8.8 g/cc, a strength of 2.1 GPa, and Poisson's ratio of 0.36. Al4Cr5Nb30Ta1Ti10V50 can have a melting temperature of 1935° C., a density of 6.8 g/cc, a strength of 2.2 GPa, and Poisson's ratio of 0.36. Al4Cr5Nb30Ta1Ti20V40 can have a melting temperature of 1912° C., a density of 6.6 g/cc, a strength of 2.2 GPa, and Poisson's ratio of 0.36. Al2Cr10Ta18Ti20V50 can have a melting temperature of 1924° C., a density of 8.0 g/cc, a strength of 2.6 GPa, and Poisson's ratio of 0.34. Al8Cr5Ta17Ti20V50 can have a melting temperature of 1913° C., a density of 7.6 g/cc, a strength of 2.4 GPa, and Poisson's ratio of 0.34. Al8Nb30Ta2Ti20V40 can have a melting temperature of 1910° C., a density of 6.5 g/cc, a strength of 2.1 GPa, and Poisson's ratio of 0.37. Al4Ni8Ti44V28W16 can have a melting temperature of 1965° C., a density of 7.5 g/cc, a strength of 2.6 GPa, and Poisson's ratio of 0.33. Al2Nb24Ni8Ti22V44 can have a melting temperature of 1801° C., a density of 6.5 g/cc, a strength of 2.2 GPa, and Poisson's ratio of 0.36. Al6Ni8Ti26V44W16 can have a melting temperature of 1983° C., a density of 8.9 g/cc, a strength of 2.5 GPa, and Poisson's ratio of 0.34. Al6Nb24Ni8Ti10V44W8 can have a melting temperature of 1987° C., a density of 8.6 g/cc, a strength of 2.3 GPa, and Poisson's ratio of 0.36. Al4Cr1Nb30Ni5Ti4V56 can have a melting temperature of 1929° C., a density of 7.6 g/cc, a strength of 2.2 GPa, and Poisson's ratio of 0.37. Al6Nb16Ni8Ti26V36W8 can have a melting temperature of 1861° C., a density of 8.1 g/cc, a strength of 2.3 GPa, and Poisson's ratio of 0.35. Al6Cr6Ni8Ti28V36W16 can have a melting temperature of 1981° C., a density of 8.9 g/cc, a strength of 2.6 GPa, and Poisson's ratio of 0.32. Al2Nb16Ni8Ti14V44W16 can have a melting temperature of 2082° C., a density of 8.6 g/cc, a strength of 2.6 GPa, and Poisson's ratio of 0.35. Al2Mo8Nb24Ni8Ti22V36 can have a melting temperature of 1871° C., a density of 6.8 g/cc, a strength of 2.0 GPa, and Poisson's ratio of 0.36. Al2Cr12Nb16Ni8Ti10V44W8 can have a melting temperature of 1921° C., a density of 7.7 g/cc, a strength of 2.6 GPa, and Poisson's ratio of 0.34. Al2Hf8Nb24Ni8Ti14V36W8 can have a melting temperature of 1962° C., a density of 8.5 g/cc, a strength of 2.3 GPa, and Poisson's ratio of 0.36.

Besides HEA solid solutions and composites, the alloy design framework can be adapted to discover new functional intermetallic compounds. The goal is to achieve significant improvement in thermoelectric, magnetic, and electrical and thermal properties, just to name a few. Current functional materials design is primarily based on computation-intensive first-principles calculations. ML can accelerate the design process. However, existing functional HEAs have limited datasets for ML training applications. This shortcoming can be overcome by using the inventive method disclosed herein. The joint use of primary and adaptive features, along with physics-based features can provide the prediction and expand the database as outlined in the following:
Primary features will explore the solid solubility boundaries of the sublattices using binary alloy phase diagrams.
Adaptive features will evaluate the synthesizability by considering the degree of mixing enthalpies mismatch between the sublattices. The atomic size mismatch can be considered as needed.
Physics-based features will be formulated using physical parameters that best characterize the properties.

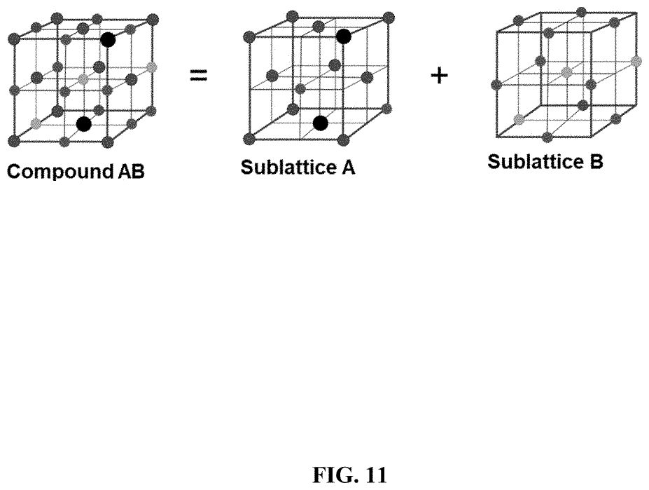

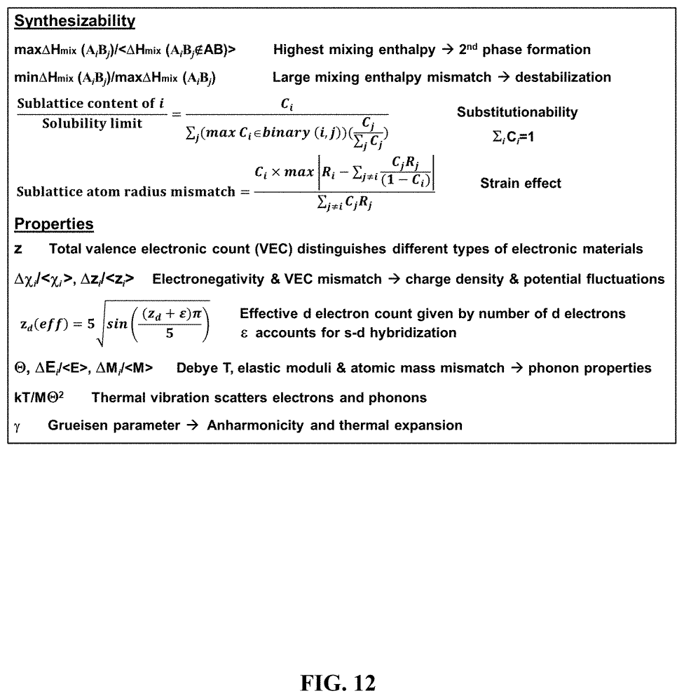

FIG. 11 shows crystal structures of a hypothetical high-entropy intermetallic compound based on A₄B₄ and its two sublattices A and B. The alloy design framework can be used to predict the synthesizability and electronic properties of a hypothetical high-entropy intermetallic compound A₄B₄. The crystal structure of A₄B₄ is shown in FIG. 11. Candidate ML features are constructed by considering the substitutional ability of the elemental components, which determines the synthesizability of A₄B₄ while also allowing properties design. Note that the A₄B₄ compound referred to here is used for illustration purposes. The features formulated can be applied to many different types of compounds. At the basic level, the substitutability of the two sublattices is determined by two factors. The first factor is solute solubility in each of the sublattices, which can be inferred from the solubility limits found in the binary alloy phase diagrams. The second factor is the robustness of the crystal structure, which can be considered from the perspectives of mixing enthalpy mismatch and the degree of lattice mismatch causing strain. A large mixing enthalpy mismatch or lattice strain can destabilize the crystal structure, resulting in phase separation or phase transformation. These ML features for predicting synthesizability are given in FIG. 12.

The functional properties are designed jointly with synthesizability. Several physics-based features are identified and listed in FIG. 12. In general, the ratio Δxᵢ/<xᵢ> denotes mismatch in the elemental parameter xᵢ. Other parameters include x the electronegativity, z the total valence electron count, zᵢ the elemental valence, and z_{d} the number of d electrons per atom. z plays an important role in the classification of semiconductors since the occurrence of bandgap usually follows a certain valence rule. The expression for the effective valence d electron count takes s-d hybridization into account. The latter is characterized by a parameter ε (assumed to be less than 0.5) such that the effect of the d band does not automatically vanish when z_{d} is 0, 5, or 10, that is when the d band empty, half filled, and fully filled, respectively. The d band influences the material properties in an important way through its high effective mass. For the physics-based features, the various mismatches Δxᵢ/<xᵢ> shown in FIG. 12 infer local fluctuations in charge density, interatomic interaction, and elasticity, all of which can influence the electronic and vibrational properties.

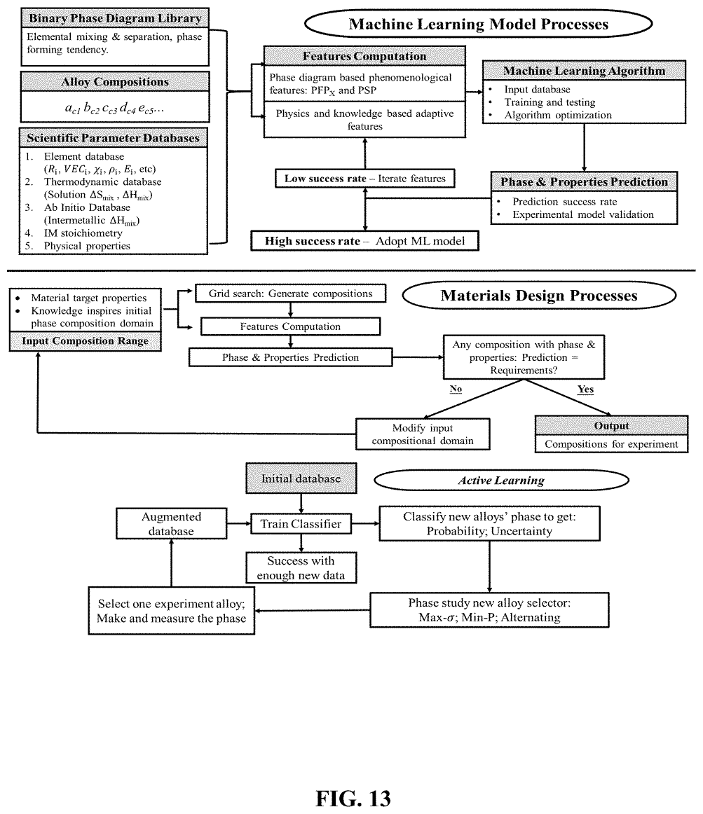

The alloy design software can include two main computational modules, namely: “Machine Learning Model Processes” and “Materials Design Processes” that can be used either separately or jointly depending on the objective. The flowchart in FIG. 13 illustrates the flow of processes within each of the modules. Each module has several operation algorithms for specific tasks such as phase diagram image scanning, features computation, data training, and testing, prediction and optimization, as well as active learning. The modules can provide the following service functions:
Machine Learning Model Processes—This module includes of the processes for establishing a machine learning model for predicting and validating the material's phases and properties. Algorithms are written to scan the relevant binary phase diagrams, extract the information from input databases, and compute the feature values. Different machine learning algorithms are used in training and testing the dataset. Based on the outcome, one of the algorithms will be selected to predict alloy phases and properties. The predicted results and associated uncertainties are obtained. This process is iterated, and the optimized ML is obtained.
Materials Design Processes—This module can be used for alloy design, particularly for “inverse design”. In inverse design, certain property is desired, and one is asked to search for the alloy composition and phase. This design problem has no general solution. Based on prior knowledge, one can conceive a compositional domain and input it into the prior established machine learning model to produce the initial result. The yield compositions can be iterated in a closed-loop fashion to produce the desired phase. This module can have a sub-module for active learning. When the ML database is small, active learning can wisely select new compositions, as few as possible, to prepare, characterize, and add into the database. A bootstrapping technique can be applied to return the ML classification probability and uncertainty when predicting a new alloy. Then the next experiment alloy can be selected from all the new alloy candidates using strategies include: (1) Max-σ: select the experiment with the highest prediction uncertainty³⁵; (2) Min-P: select the experiment with the lowest prediction probability²⁷ (3) Alternating between the previous two selectors. The selector's performance can be evaluated by the ML cross-validation accuracy with the updated database after certain rounds of new experiments. The best selector, which leads to a higher ML accuracy improvement, can further improve the model.
The software can be utilized to combine phase formation and material properties. The computed material properties can include mechanical properties, such as Poisson's ratio, density, strength, and functional properties such as corrosion resistance, thermal and thermoelectric properties, and magnetic properties. The related cost estimate can be conveniently incorporated into the software.

Embodiments can relate to a method for predicting thermodynamic phase of a material. The method can involve obtaining a binary phase diagram for each material to be used as a component of a high-entropy alloy (HEA). The method can involve generating a primary feature that is representative of a probability that the HEA will exhibit a solid solution phase and/or an intermetallic phase. The method can involve generating an adaptive feature that is representative of a factor favoring formation of a desired intermetallic HEA phase. The method can involve encoding the primary feature and/or the adaptive feature with thermodynamic data associated with formation of HEA alloy phases. The method can involve generate an output representation of the HEA alloy phases for a material under analysis.

In some embodiments, the method can involve generating a compositional space plot for the HEA alloy phases. The compositional space plot can be a representation of the HEA alloy phases.

The method can involve defining a temperature-composition region for the primary feature that is a region on a binary phase diagram bounded by a melting temperature T. and a phase formation temperature T_{pf}.

The method can involve generating the primary feature and/or the adaptive feature is performed using machine learning techniques.

The method can involve optimizing the primary feature and/or the adaptive feature via sequential training.

Each elemental component has specific functionality in a multi-component alloy. The high-entropy concept of diverse chemistry and complex composition provides the opportunity for realizing unprecedented material properties. The invention alloy design framework exploits this opportunity to predict a new class of alloys to deliver translational successes. The design framework can be implemented and practiced through a design software package in accelerating technology transfer in several application areas. Examples are the following:
Turbine blades—Current Ni-based superalloys used in stage-one gas-turbine blades have limited high-temperature fatigue and corrosion resistance properties capping the combustor firing temperature and thus limiting the combined cycle efficiency of the engine. High-temperature refractory HEAs can be designed to overcome this limitation.
Light-weight materials—High-entropy alloys can be designed as body frames for cars and airplanes to increase the strength-to-weight ratio in improving energy efficiency.
Corrosion resistance coating—Great flexibility in selecting passivation elements to produce suitable oxide inclusions and various kinds of oxides in increasing the corrosion resistance.
Thermoelectric generation and refrigeration—The high-entropy alloys approach allows the electronic and thermal properties to be tuned more easily. Some examples include power generation by recovering waste heat, small-scale refrigerators for storage, and cryo-coolers for medical imaging and scientific research.
Magnetic cooling—High-entropy approach can be employed to tune the magnetic entropy and peak temperatures to improve the coefficient of performance in magnetocaloric cooling technology.
Thermal coatings—High-entropy ceramics designed using a similar concept can provide thermal barriers for protection in high-temperature environment.
Medical implants—High-entropy alloys with high strength to density ratio and environmental resilience can potentially outperform current nickel-chromium alloys.

The following references are incorporated herein by reference in their entireties.
1. Cantor, B., Chang, I. T. H., Knight, P. & Vincent, A. J. B. Microstructural development in equiatomic multi-component alloys. Mater. Sci. Eng. A 375-377, 213-218 (2004).
2. Yeh, J. W. et al. Nanostructured high-entropy alloys with multiple principal elements: Novel alloy design concepts and outcomes. Adv. Eng. Mater. 6, 299-303 (2004).
3. Miracle, D. B. & Senkov, O. N. A critical review of high entropy alloys and related concepts. Acta Mater. 122, 448-511 (2017).
4. Li, Z., Zhao, S., Ritchie, R. O. & Meyers, M. A. Mechanical properties of high-entropy alloys with emphasis on face-centered cubic alloys. Prog. Mater. Sci. 102, 296-345 (2019).
5. Shi, P. et al. Enhanced strength—ductility synergy in ultrafine-grained eutectic high-entropy alloys by inheriting microstructural lamellae. Nat. Commun. 10, 1-8 (2019).
6. Lu, P. et al. Computational design and initial corrosion assessment of a series of non-equimolar high entropy alloys. Scr. Mater. 172, 12-16 (2019).
7. Yao, Y. et al. Carbothermal shock synthesis of high-entropy-alloy nanoparticles. Science (80-.). 359, 1489-1494 (2018).
8. Braun, J. L. et al. Charge-induced disorder controls the thermal conductivity of entropy-stabilized oxides. Adv. Mater. 30, 1-8 (2018).
9. Liu, R. et al. Entropy as a Gene-Like Performance Indicator Promoting Thermoelectric Materials. Adv. Mater. 29, 1-7 (2017).
10. Poon, S. J. & He, J. Multi-Principal-Element Approach to High-Performance Thermoelectric Materials. Ref. Modul. Mater. Sci. Mater. Eng. (2019). doi:10.1016/B978-0-12-803581-8.11719-9
11. Toda-Caraballo, I. & Rivera-Diaz-Del-Castillo, P. E. J. A criterion for the formation of high entropy alloys based on lattice distortion. Intermetallics 71, 76-87 (2016).
12. Gao, M. C. et al. Thermodynamics of concentrated solid solution alloys. Curr. Opin. Solid State Mater. Sci. 21, 238-251 (2017).
13. Ikeda, Y., Grabowski, B. & Kormann, F. Ab initio phase stabilities and mechanical properties of multi-component alloys: A comprehensive review for high entropy alloys and compositionally complex alloys. Materials Characterization 147, 464-511 (2019).
14. Tian, F. et al. Structural stability of NiCoFeCrAlx high-entropy alloy from ab initio theory. Phys. Rev. B—Condens. Matter Mater. Phys. 88, 1-5 (2013).
15. Senkov, O. N., Miller, J. D., Miracle, D. B. & Woodward, C. Accelerated exploration of multi-principal element alloys for structural applications. Calphad Comput. Coupling Phase Diagrams Thermochem. 50, 32-48 (2015).
16. Senkov, O. N. et al. CALPHAD-aided development of quaternary multi-principal element refractory alloys based on NbTiZr. J. Alloys Compd. 783, 729-742 (2019).
17. Kube, S. A. et al. Phase selection motifs in High Entropy Alloys revealed through combinatorial methods: Large atomic size difference favors BCC over FCC. Acta Mater. 166, 677-686 (2019).
18. Tancret, F., Toda-Caraballo, I., Menou, E. & Rivera Diaz-Del-Castillo, P. E. J. Designing high entropy alloys employing thermodynamics and Gaussian process statistical analysis. Mater. Des. 115, 486-497 (2017).
19. Huang, W., Martin, P. & Zhuang, H. L. Machine-learning phase prediction of high-entropy alloys. Acta Mater. 169, 225-236 (2019).
20. Islam, N., Huang, W. & Zhuang, H. L. Machine learning for phase selection in multi-principal element alloys. Comput. Mater. Sci. 150, 230-235 (2018).
21. Chang, Y. J., Jui, C. Y., Lee, W. J. & Yeh, A. C. Prediction of the Composition and Hardness of High-Entropy Alloys by Machine Learning. Jom 71, 3433-3442 (2019).
22. Wen, C. et al. Machine learning assisted design of high entropy alloys with desired property. Acta Mater. 170, 109-117 (2019).
23. Pei, Z., Yin, J., Hawk, J. A., Alman, D. E. & Gao, M. C. Machine-learning informed prediction of high-entropy solid solution formation: Beyond the Hume-Rothery rules. npj Comput. Mater. (2020). doi:10.13140/RG.2.2.32615.19360
24. Li, Y. & Guo, W. Machine-learning model for predicting phase formations of high-entropy alloys. Phys. Rev. Mater. 3, 95005 (2019).
25. Qi, J., Cheung, A. M. & Poon, S. J. High Entropy Alloys Mined From Binary Phase Diagrams. Sci. Rep. 9, 1-10 (2019).
26. Dai, D. et al. Using machine learning and feature engineering to characterize limited material datasets of high-entropy alloys. Comput. Mater. Sci. 175, (2020).
27. Zhang, Y. et al. Phase prediction in high entropy alloys with a rational selection of materials descriptors and machine learning models. Acta Mater. 185, 528-539 (2020).
28. Li, C. et al. Microstructures and mechanical properties of body-centered-cubic (Al,Ti)0.7(Ni,Co,Fe,Cr)5 high entropy alloys with coherent B2/L21 nanoprecipitation. Mater. Sci. Eng. A 737, 286-296 (2018).
29. Qi, J., Cheung, A. M. & Poon, S. J. Navigating the Complex Compositional Landscape of High-Entropy Alloys. (2020).
30. Graf, T., Felser, C. & Parkin, S. S. P. Simple rules for the understanding of Heusler compounds. Prog. Solid State Chem. 39, 1-50 (2011).
31. Polvani, R. S., Tzeng, W. S. & Strutt, P. R. High temperature creep in a semi-coherent NiAl—Ni2AlTi alloy. Metall. Trans. A 7, 33-40 (1976).
32. Strutt, P. R., Polvani, R. S. & Ingram, J. C. Creep behavior of the heusler type structure alloy Ni2AlTi. Metall. Trans. A 7, 23-31 (1976).
33. Liu, J., Gottschall, T., Skokov, K. P., Moore, J. D. & Gutfleisch, O. Giant magnetocaloric effect driven by structural transitions. Nat. Mater. 11, 620-626 (2012).
34. Miracle, D. B., Tsai, M. H., Senkov, O. N., Soni, V. & Banerjee, R. Refractory high entropy superalloys (RSAs). Scr. Mater. 187, 445-452 (2020).
35. Balachandran, P. V., Xue, D., Theiler, J., Hogden, J. & Lookman, T. Adaptive Strategies for Materials Design using Uncertainties. Sci. Rep. 6, 1-9 (2016).

It will be understood that modifications to the embodiments disclosed herein can be made to meet a particular set of design criteria. For instance, any of the components discussed herein can be any suitable number or type of each to meet a particular objective. Therefore, while certain exemplary embodiments of the system 100 and methods of making and using the same disclosed herein have been discussed and illustrated, it is to be distinctly understood that the invention is not limited thereto but can be otherwise variously embodied and practiced within the scope of the following claims.

It will be appreciated that some components, features, and/or configurations can be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein. Additionally, the disclosure of a range of values is a disclosure of every numerical value within that range, including the end points.

What is claimed is:

1. A system for predicting thermodynamic phase of a material, the system comprising:
a processor in operative association with memory, the processor including plural processing modules, wherein:
a phase diagram image scanning processing module is configured to scan a binary phase diagram for each material to be used as a component of a high-entropy alloy (REA);
a feature computation processing module configured to generate a primary feature and an adaptive feature, wherein:
the primary feature is representative of a probability that the HEA will exhibit a solid solution phase and/or an intermetallic phase, the primary feature including:
a phase field parameter (PFPₓ) that is representative of a probability of forming phase X for the whole HEA; and
a phase separation percentage (PSP) that is representative of a probability that two elements of the HEA will be separated into two different phases;
the adaptive feature is representative of a factor favoring formation of a desired intermetallic HEA phase, the factor including any one or combination of:
a threshold mixing enthalpy indicating that more than one type of phase formation is possible;
a threshold of total atomic percentage of components in the HEA that favors dissolution of the components in the HEA in a solid solution;
a threshold ratio of concentration of phase forming elements to total atomic percentage that favors precipitation of a phase;
a threshold weighted electronegativity ratio that favors formation of a phase;
a threshold mixing entropy that favors disordered phase formation; or
a threshold ratio of a desired element content to all transitional element content that favors formation of a phase;
a prediction module configured to encode the primary feature and/or the adaptive feature with thermodynamic data associated with formation of HEA alloy phases to provide an output representation of the HEA alloy phases for a material under analysis.

2. The system of claim 1, wherein:
the prediction module is configured to generate as the output a compositional space plot for the HEA alloy phases, the compositional space plot being a representation of the HEA alloy phases.

3. The system of claim 1, wherein:
the feature computation processing module is configured to define a temperature-composition region for the primary feature that is a region on a binary phase diagram bounded by a melting temperature Tₘ and a phase formation temperature T_{pf}.

4. The system of claim 3, wherein:
$${T}_{m} = \frac{{\sum }_{i \neq j} {T}_{i - j} \times {c}_{i} \times {c}_{j}}{{\sum }_{i \neq j} {c}_{i} \times {c}_{j}} ,$$
where Tᵢ₋ⱼ is the binary liquidus temperatures on the binary phase diagram of i-j elements when a relative ratio of two elements of the binary phase diagram is cᵢ:cⱼ.

5. The system of claim 4, wherein:
T_{pf}≈0.8 Tₘ.

6. The system of claim 5, wherein:
$${PFP}_{X} = \frac{{\sum }_{i \neq j} {X}_{i - j} \times {c}_{i} \times {c}_{j}}{{\sum }_{i \neq j} {c}_{i} \times {c}_{j}} \div 100 \% .$$

7. The system of claim 1, wherein:
the feature computation processing module is configured to determine a PFPₓ for any one or combination of:
PFP_{A1}, which is representative of an A1 (FCC) phase;
PFP_{A2}, which is representative of an A2 (BCC) phase;
PFP_{B2}, which is representative of an Al—(Ni, Fe, Co) type B2 phase;
PFP_{A3}, which is representative of an A3 (hexagonal) phase;
PFP_{Laves}, which is representative of a Laves phase; or
PFP_{Sigma}, which is representative of a Sigma phase.

8. The system of claim 5, wherein:
$$PSP = \frac{{\sum }_{i \neq j} {Separation}_{i - j} \times {c}_{i} \times {c}_{j}}{{\sum }_{i \neq j} {Mixing}_{i - j} \times {c}_{i} \times {c}_{j}} ,$$
where Separationᵢ₋ⱼ and Mixingᵢ₋ⱼ are binary phase separation percentage and mixing percentage, respectively, between i-j element pair.

9. The system of claim 8, wherein:
a combined total of Separationᵢ₋ⱼ and Mixingᵢ₋ⱼ is 100%.

10. The system of claim 8, wherein:
Separationᵢ₋ⱼ=0% when the phase separation is absent from a binary phase diagram.

11. The system of claim 1, wherein:
the feature computation processing module is configured to generate the primary feature and/or the adaptive feature using machine learning techniques.

12. The system of claim 11, wherein:
the feature computation processing module is configured to optimize the primary feature and/or the adaptive feature via sequential training.

13. A high-entropy alloy, comprising any one of:
Al3Nb47Ta18Ti20V12; Al6Nb50Ta12Ti20V6W6; Al9Nb47Ta12Ti20V6W6; Al3Nb41Ti20V18W6Zr12; Nb50Ta12Ti20W6Zr12; Nb32Ta18Ti20V24Zr6; Nb32Ti20V24W12Zr12; Al3Hf6Nb35Ta12Ti20V24; Al3Nb41Ta12Ti20V18Zr6; Al3Nb47Ta18Ti20Zr12; Al9Hf6Nb41Ti20V18W6; Al6Nb32Ta18Ti20V24; Al6Nb26Ta12Ti20V30Zr6; Al3Nb41Ta12Ti20V18Zr6; Al6Nb48Ta12Ti10W6Zr18; Al9Nb29Ti20V30W6Zr6; Al3Nb42Ta21Ti20Zr14; Al6Nb39Ta21Ti20Zr14; Nb50Ta12Ti20W6Zr12; Cr5Hf6Nb48Ta7Ti20Zr14; Cr10Hf6Nb43Ta14Ti20Zr7; Cr15Hf6Nb43Ti15Zr21; Cr15Hf6Nb41Ti10Zr28; Cr10Nb49Ta14Ti20Zr7; Cr5Nb47Ta14Ti20Zr14; Nb45Ta14Ti20Zr21; Nb38Ta21Ti20Zr21; Al6Cr5Nb39Ta14Ti15Zr21; Al9Nb36Ta21Ti20Zr14; Al9Cr15Nb34Ta14Zr28; Al9Nb29Ni15Ta14Ti5Zr28; Al3Nb33Ni5Ta21Ti10Zr28; Al3Nb49Ni5Ta14Ti15Zr14; Al6Nb46Ni15Ta14Ti5Zr14; Al4Cr5Nb30Ta1Ti10V50; Al4Cr5Nb30Ta1Ti20V40; Al2Cr10Ta18Ti20V50; Al8Cr5Ta17Ti20V50; Al8Nb30Ta2Ti20V40; Al4Ni8Ti44V28W16; Al2Nb24Ni8Ti22V44; Al6Ni8Ti26V44W16; Al6Nb24Ni8Ti10V44W8; Al4Cr1Nb30Ni5Ti4V56; Al6Nb16Ni8Ti26V36W8; Al6Cr6Ni8Ti28V36W16; Al2Nb16Ni8Ti14V44W16; Al2Mo8Nb24Ni8Ti22V36; Al2Cr12Nb16Ni8Ti10V44W8; or Al2Hf8Nb24Ni8Ti14V36W8.

14. The high-entropy alloy of claim 13, wherein:
Al3Nb47Ta18Ti20V12 has a BBC phase;
Al6Nb50Ta12Ti20V6W6 has a BBC phase;
Al9Nb47Ta12Ti20V6W6 has a BBC phase;
Al3Nb41Ti20V18W6Zr12 has a BBC phase;
Nb50Ta12Ti20W6Zr12 has a BBC phase;
Nb32Ta18Ti20V24Zr6 has a BBC phase;
Nb32Ti20V24W12Zr12 has a BBC phase;
Al3Hf6Nb35Ta12Ti20V24 has a BBC phase;
Al3Nb41Ta12Ti20V18Zr6 has a BBC phase;
Al3Nb47Ta18Ti20Zr12 has a BBC phase;
Al9Hf6Nb41Ti20V18W6 has a BBC+B2 phase;
Al6Nb32Ta18Ti20V24 has a BBC+B2 phase;
Al6Nb26Ta12Ti20V30Zr6 has a BBC+B2 phase;
Al3Nb41Ta12Ti20V18Zr6 has a BBC+B2 phase;
Al6Nb48Ta12Ti10W6Zr18 has a BBC+B2 phase;
Al9Nb29Ti20V30W6Zr6 has a BBC+B2 phase;
Al3Nb42Ta21Ti20Zr14 has a BBC phase;
Al6Nb39Ta21Ti20Zr14 has a BBC phase;
Nb50Ta12Ti20W6Zr12 has a BBC phase;
Cr5Hf6Nb48Ta7Ti20Zr14 has a BBC phase;
Cr10Hf6Nb43Ta14Ti20Zr7 has a BBC phase;
Cr15Hf6Nb43Ti15Zr21 has a BBC phase;
Cr15Hf6Nb41Ti10Zr28 has a BBC phase;
Cr10Nb49Ta14Ti20Zr7 has a BBC phase;
Cr5Nb47Ta14Ti20Zr14 has a BBC phase;
Nb45Ta14Ti20Zr21 has a BBC phase;
Nb38Ta21Ti20Zr21 has a BBC phase;
Al6Cr5Nb39Ta14Ti15Zr21 has a BBC+B2 phase;
Al9Nb36Ta21Ti20Zr14 has a BBC+B2 phase;
Al9Cr15Nb34Ta14Zr28 has a BBC+B2 phase;
Al9Nb29Ni15Ta14Ti5Zr28 has a BBC+L21 phase;
Al3Nb33Ni5Ta21Ti10Zr28 has a BBC+L21 phase;
Al3Nb49Ni5Ta14Ti15Zr14 has a BBC+L21 phase;
Al6Nb46Ni15Ta14Ti5Zr14 has a BBC+L21 phase;
Al4Cr5Nb30Ta1Ti10V50 has a BBC phase;
Al4Cr5Nb30Ta1Ti20V40 has a BBC phase;
Al2Cr10Ta18Ti20V50 has a BBC phase;
Al8Cr5Ta17Ti20V50 has a BBC phase;
Al8Nb30Ta2Ti20V40 has a BBC phase;
Al4Ni8Ti44V28W16 has a BBC+L21 phase;
Al2Nb24Ni8Ti22V44 has a BBC+L21 phase;
Al6Ni8Ti26V44W16 has a BBC+L21 phase;
Al6Nb24Ni8Ti10V44W8 has a BBC+L21 phase;
Al4Cr1Nb30Ni5Ti4V56 has a BBC+L21 phase;
Al6Nb16Ni8Ti26V36W8 has a BBC+L21 phase;
Al6Cr6Ni8Ti28V36W16 has a BBC+L21 phase;
Al2Nb16Ni8Ti14V44W16 has a BBC+L21 phase;
Al2Mo8Nb24Ni8Ti22V36 has a BBC+L21 phase;
Al2Cr12Nb16Ni8Ti10V44W8 has a BBC+L21 phase; and
Al2Hf8Nb24Ni8Ti14V36W8 has a BBC+L21 phase.

15. The high-entropy alloy of claim 14 designed for high thermal stability, ductility, and high strengths, wherein:
Al3Nb47Ta18Ti20V12 has a melting temperature of 2299° C., a density of 8.9 g/cc, a strength of 2.1 GPa, and Poisson's ratio of 0.37;
Al6Nb50Ta12Ti20V6W6 has a melting temperature of 2329° C., a density of 8.9 g/cc, a strength of 2.1 GPa, and Poisson's ratio of 0.36;
Al9Nb47Ta12Ti20V6W6 has a melting temperature of 2288° C., a density of 8.7 g/cc, a strength of 2.1 GPa, and Poisson's ratio of 0.36;
Al3Nb41Ti20V18W6Zr12 has a melting temperature of 2079° C., a density of 7.5 g/cc, a strength of 2.1 GPa, and Poisson's ratio of 0.36;
Nb50Ta12Ti20W6Zr12 has a melting temperature of 2288° C., a density of 9.0 g/cc, a strength of 2.1 GPa, and Poisson's ratio of 0.36;
Nb32Ta18Ti20V24Zr6 has a melting temperature of 2141° C., a density of 8.6 g/cc, a strength of 2.2 GPa, and Poisson's ratio of 0.36;
Nb32Ti20V24W12Zr12 has a melting temperature of 2106° C., a density of 8.1 g/cc, a strength of 2.2 GPa, and Poisson's ratio of 0.35;
Al3Hf6Nb35Ta12Ti20V24 has a melting temperature of 2094° C., a density of 8.5 g/cc, a strength of 2.1 GPa, and Poisson's ratio of 0.37;
Al3Nb41Ta12Ti20V18Zr6 has a melting temperature of 2149° C., a density of 8.1 g/cc, a strength of 2.1 GPa, and Poisson's ratio of 0.37;
Al3Nb47Ta18Ti20Zr12 has a melting temperature of 2276° C., a density of 8.8 g/cc, a strength of 2.0 GPa, and Poisson's ratio of 0.36;
Al9Hf6Nb41Ti20V18W6 has a melting temperature of 2109° C., a density of 7.8 g/cc, a strength of 2.0 GPa, and Poisson's ratio of 0.36;
Al6Nb32Ta18Ti20V24 has a melting temperature of 2152° C., a density of 8.5 g/cc, a strength of 2.2 GPa, and Poisson's ratio of 0.36;
Al6Nb26Ta12Ti20V30Zr6 has a melting temperature of 1998° C., a density of 7.6 g/cc, a strength of 2.1 GPa, and Poisson's ratio of 0.36;
Al3Nb41Ta12Ti20V18Zr6 has a melting temperature of 2149° C., a density of 8.1 g/cc, a strength of 2.1 GPa, and Poisson's ratio of 0.37;
Al6Nb48Ta12Ti10W6Zr18 has a melting temperature of 2280° C., a density of 8.9 g/cc, a strength of 2.0 GPa, and Poisson's ratio of 0.36;
Al9Nb29Ti20V30W6Zr6 has a melting temperature of 1985° C., a density of 7.0 g/cc, a strength of 2.1 GPa, and Poisson's ratio of 0.36;
Al3Nb42Ta21Ti20Zr14 has a melting temperature of 2275° C., a density of 8.9 g/cc, a strength of 2.1 GPa, and Poisson's ratio of 0.36;
Al6Nb39Ta21Ti20Zr14 has a melting temperature of 2246° C., a density of 8.8 g/cc, a strength of 2.0 GPa, and Poisson's ratio of 0.36;
Nb50Ta12Ti20W6Zr12 has a melting temperature of 2288° C., a density of 9.0 g/cc, a strength of 2.1 GPa, and Poisson's ratio of 0.36;
Cr5Hf6Nb48Ta7Ti20Zr14 has a melting temperature of 2145° C., a density of 8.3 g/cc, a strength of 2.0 GPa, and Poisson's ratio of 0.36;
Cr10Hf6Nb43Ta14Ti20Zr7 has a melting temperature of 2199° C., a density of 9.0 g/cc, a strength of 2.2 GPa, and Poisson's ratio of 0.35;
Cr15Hf6Nb43Ti15Zr21 has a melting temperature of 2014° C., a density of 7.7 g/cc, a strength of 2.0 GPa, and Poisson's ratio of 0.34;
Cr15Hf6Nb41Ti10Zr28 has a melting temperature of 1991° C., a density of 7.7 g/cc, a strength of 2.0 GPa, and Poisson's ratio of 0.34;
Cr10Nb49Ta14Ti20Zr7 has a melting temperature of 2222° C., a density of 8.6 g/cc, a strength of 2.2 GPa, and Poisson's ratio of 0.35;
Cr5Nb47Ta14Ti20Zr14 has a melting temperature of 2202° C., a density of 8.5 g/cc, a strength of 2.1 GPa, and Poisson's ratio of 0.36;
Nb45Ta14Ti20Zr21 has a melting temperature of 2164° C., a density of 8.4 g/cc, a strength of 2.0 GPa, and Poisson's ratio of 0.36;
Nb38Ta21Ti20Zr21 has a melting temperature of 2203° C., a density of 8.9 g/cc, a strength of 2.1 GPa, and Poisson's ratio of 0.36;
Al6Cr5Nb39Ta14Ti15Zr21 has a melting temperature of 2129° C., a density of 8.2 g/cc, a strength of 2.0 GPa, and Poisson's ratio of 0.35;
Al9Nb36Ta21Ti20Zr14 has a melting temperature of 2209° C., a density of 8.6 g/cc, a strength of 2.0 GPa, and Poisson's ratio of 0.36;
Al9Cr15Nb34Ta14Zr28 has a melting temperature of 1994° C., a density of 8.3 g/cc, a strength of 2.1 GPa, and Poisson's ratio of 0.34;
Al9Nb29Ni15Ta14Ti5Zr28 has a melting temperature of 1882° C., a density of 8.3 g/cc, a strength of 2.0 GPa, and Poisson's ratio of 0.35;
Al3Nb33Ni5Ta21Ti10Zr28 has a melting temperature of 2169° C., a density of 9.0 g/cc, a strength of 2.1 GPa, and Poisson's ratio of 0.36;
Al3Nb49Ni5Ta14Ti15Zr14 has a melting temperature of 2221° C., a density of 8.6 g/cc, a strength of 2.0 GPa, and Poisson's ratio of 0.36;
Al6Nb46Ni15Ta14Ti5Zr14 has a melting temperature of 2128° C., a density of 8.8 g/cc, a strength of 2.1 GPa, and Poisson's ratio of 0.36;
Al4Cr5Nb30Ta1Ti10V50 has a melting temperature of 1935° C., a density of 6.8 g/cc, a strength of 2.2 GPa, and Poisson's ratio of 0.36;
Al4Cr5Nb30Ta1Ti20V40 has a melting temperature of 1912° C., a density of 6.6 g/cc, a strength of 2.2 GPa, and Poisson's ratio of 0.36;
Al2Cr10Ta18Ti20V50 has a melting temperature of 1924° C., a density of 8.0 g/cc, a strength of 2.6 GPa, and Poisson's ratio of 0.34;
Al8Cr5Ta17Ti20V50 has a melting temperature of 1913° C., a density of 7.6 g/cc, a strength of 2.4 GPa, and Poisson's ratio of 0.34;
Al8Nb30Ta2Ti20V40 has a melting temperature of 1910° C., a density of 6.5 g/cc, a strength of 2.1 GPa, and Poisson's ratio of 0.37;
Al4Ni8Ti44V28W16 has a melting temperature of 1965° C., a density of 7.5 g/cc, a strength of 2.6 GPa, and Poisson's ratio of 0.33;
Al2Nb24Ni8Ti22V44 has a melting temperature of 1801° C., a density of 6.5 g/cc, a strength of 2.2 GPa, and Poisson's ratio of 0.36;
Al6Ni8Ti26V44W16 has a melting temperature of 1983° C., a density of 8.9 g/cc, a strength of 2.5 GPa, and Poisson's ratio of 0.34;
Al6Nb24Ni8Ti10V44W8 has a melting temperature of 1987° C., a density of 8.6 g/cc, a strength of 2.3 GPa, and Poisson's ratio of 0.36;
Al4Cr1Nb30Ni5Ti4V56 has a melting temperature of 1929° C., a density of 7.6 g/cc, a strength of 2.2 GPa, and Poisson's ratio of 0.37;
Al6Nb16Ni8Ti26V36W8 has a melting temperature of 1861° C., a density of 8.1 g/cc, a strength of 2.3 GPa, and Poisson's ratio of 0.35;
Al6Cr6Ni8Ti28V36W16 has a melting temperature of 1981° C., a density of 8.9 g/cc, a strength of 2.6 GPa, and Poisson's ratio of 0.32;
Al2Nb16Ni8Ti14V44W16 has a melting temperature of 2082° C., a density of 8.6 g/cc, a strength of 2.6 GPa, and Poisson's ratio of 0.35;
Al2Mo8Nb24Ni8Ti22V36 has a melting temperature of 1871° C., a density of 6.8 g/cc, a strength of 2.0 GPa, and Poisson's ratio of 0.36;
Al2Cr12Nb16Ni8Ti10V44W8 has a melting temperature of 1921° C., a density of 7.7 g/cc, a strength of 2.6 GPa, and Poisson's ratio of 0.34; and
Al2Hf8Nb24Ni8Ti14V36W8 has a melting temperature of 1962° C., a density of 8.5 g/cc, a strength of 2.3 GPa, and Poisson's ratio of 0.36.

16. A method for predicting thermodynamic phase of a material, the method comprising:
obtaining a binary phase diagram for each material to be used as a component of a high-entropy alloy (HEA);
generating a primary feature that is representative of a probability that the HEA will exhibit a solid solution phase and/or an intermetallic phase;
generating an adaptive feature that is representative of a factor favoring formation of a desired intermetallic HEA phase;
encoding the primary feature and/or the adaptive feature with thermodynamic data associated with formation of HEA alloy phases; and
generate an output representation of the HEA alloy phases for a material under analysis.

17. The method of claim 16, comprising:
generating a compositional space plot for the HEA alloy phases, the compositional space plot being a representation of the HEA alloy phases.

18. The method of claim 16, comprising:
defining a temperature-composition region for the primary feature that is a region on a binary phase diagram bounded by a melting temperature Tₘ and a phase formation temperature T_{pf}.

19. The method of claim 16, wherein:
generating the primary feature and/or the adaptive feature is performed using machine learning techniques.

20. The method of claim 19, comprising:
optimizing the primary feature and/or the adaptive feature via sequential training.
